(19) United States
(12) Patent Application Publication
(10) Pub. No.: US 2012/0053599 A1
(43) Pub. Date: Mar. 1, 2012

(54) SUTURING, CRIMPING AND CUTTING DEVICE

(76) Inventors: Oleg Shikhman, Trumbull, CT (US); Paul A. Scirica, Huntington, CT (US)

(21) Appl. No.: 13/224,418

(22) Filed: Sep. 2, 2011

Related U.S. Application Data
(63) Continuation-in-part of application No. 10/726,034, filed on Dec. 1, 2003.
Application No. 10/726,034 is a continuation-in-part of application No. 10/726,031, filed on Dec. 1, 2003.
Application No. 10/726,031 is a continuation-in-part of application No. 10/277,794, filed on Oct. 22, 2002, now Pat. No. 8,012,165.
Application No. 10/277,794 is a continuation-in-part of application No. 10/277,793, filed on Oct. 22, 2002.
Application No. 10/277,793 is a continuation-in-part of application No. 12/098,665, filed on Apr. 7, 2008.
(60) Provisional application No. 60/343,786, filed on Oct. 22, 2001.
(60) Provisional application No. 60/345,212, filed on Oct. 22, 2001.
(60) Provisional application No. 60/922,344, filed on Apr. 6, 2007.

(51) Int. Cl. A61B 17/04

(52) U.S. Cl. 606/144

(57) ABSTRACT

A percutaneous surgical device is provided, which comprises a crimping and cutting device. The crimping and cutting portion may be located to the wound site to apply a fastener (e.g., a ferrule).

### CROSS-REFERENCE TO RELATED APPLICATIONS

The present application is a Continuation-In-Part of and claims priority to U.S. patent application Ser. No. 10/726,034, filed Dec. 1, 2003; U.S. patent application Ser. No. 10/726,031, filed Dec. 1, 2003; U.S. patent application Ser. No. 10/277,794, filed Oct. 22, 2002 which claimed priority to U.S. Provisional Application No. 60/343,786, filed Oct. 22, 2001; U.S. patent application Ser. No. 10/277,793, filed Oct. 22, 2002, which claimed priority to U.S. Provisional Application No. 60/345,212, filed Oct. 22, 2001; and, to U.S. patent application Ser. No. 12/098,665, filed Apr. 7, 2008, which claimed priority to U.S. Provisional Application No. 60/922,344, filed Apr. 6, 2007, the entire disclosures of which are specifically incorporated by reference herein.

### BACKGROUND

When performing catheterization procedures, such an angiography or angioplasty, a catheter is generally introduced percutaneously (i.e., through the skin) into the vascular system by first penetrating the skin and underlying tissue, and then the blood vessel with a sharpened hollow needle. Location of a blood vessel, such as an artery, is typically achieved by feeling for the pulse, since such structures usually cannot be seen through the skin. Next, a guide wire is commonly inserted through the lumen of the hollow needle and is caused to enter the selected blood vessel. Subsequently, the needle is typically slid off the guide wire and a combination of a dilator and sheath are fed over the guide wire and pushed through the skin to enter the vessel. The guide wire and dilator can then be removed, and the desired catheter used to carry out the procedure is fed through the lumen of the sheath and advanced through the vascular system until the working end of the catheter is appropriately positioned. Following the conclusion of the catheterization procedure, the working catheter will be withdrawn and, subsequently, the sheath can also be removed from the wound, or left in place to facilitate closure.

At this point in the procedure, the vessel leakage is controlled in order to stem the flow of blood through the puncture. Because it is common practice to administer a blood thinning agent to the patient prior to many of the catheterization procedures, stemming the blood flow can be troublesome. A common method of sealing the wound is to maintain external pressure over the vessel until the puncture naturally seals. This method of puncture closure typically takes at least thirty minutes, with the length of time usually being substantially greater if the patient is hypertensive or anti-coagulated. In some anti-coagulated patients, the sheath is left in place for hours to allow the anti-coagulant to wear off. When human hand pressure is utilized, it can be uncomfortable for the patient and can use costly professional time on the part of the hospital staff. Other pressure techniques, such as pressure bandages, sandbags or clamps, have been employed, but these devices also require the patient to remain motionless for an extended period of time and the patient must be closely monitored to ensure their effectiveness.

There remains a need in the art for effective percutaneous tissue closure that is quick, easy to instruct and easy to learn, effective and comfortable for the patient.

### SUMMARY

The above described and other disadvantages of the prior art are overcome and alleviated by the present percutaneous surgical device, which comprises a system for wound suturing and crimping and cutting device.

In one exemplary embodiment the system comprises multiple suturing devices and at least one crimping and cutting device. In such embodiment, a first suturing device may be placed within the wound to apply a first suture on edge portions across the wound site. Subsequently, a second suturing device may be placed in a different position within the wound to apply a second suture across different edge portions of the wound. Then, the crimping and cutting portion may be used to gather suture ends, and the crimping and cutting portion may be located to the wound site to apply a fastener (e.g., a ferrule).

In another exemplary embodiment, a single suturing device may be configured to apply first and second sutures on different edge portions across the wound site. In an exemplary embodiment, e.g. a dual suture delivery device may be configured similarly to single suture delivery devices with the addition of a second needle and ferrule alongside a first needle and ferrule in one or more tissue receiving gaps.

An exemplary wound suturing device comprises a housing and an elongated shaft connected thereto and at least one needle within the shaft, the at least one needle configured to travel distally across a tissue engaging gap within a tissue engaging section positioned distally from the housing on said shaft, wherein the tissue receiving gap has two opposing surfaces into which one side of a wound can be received, wherein the gap is shaped to have a depth to facilitate the placement of the edge of a wound therein such that at least one surface comprises a stop surface, wherein the stop surface is squared at a middle portion thereof to provide good tactile feel to the surgeon when tissue is engaged.

An exemplary crimping and cutting device comprises a hammer head having a first side and an opposite second side, and a ferrule engaging edge located on the second side, a tip having a distal end and a proximal end, the tip having a hammer head opening for receiving the hammer head, the hammer head opening extending from the distal end to the proximal end, the tip further having a ferrule accepting opening near the distal end, and a cutting edge within the hammer head opening of the tip, the cutting edge located proximally of the ferrule accepting opening.

The above described and other features are exemplified by the following figures and detailed description.

### BRIEF DESCRIPTION OF THE DRAWINGS

Referring now to the figures wherein the like elements are numbered alike:

FIG. 1A is a side plan view of the an exemplary wound suturing apparatus;

FIG. 2A is a bottom perspective view of an exemplary retainer member;

FIGS. 3A-5A illustrate perspective views of an exemplary actuation mechanism;

FIG. 6A is a perspective view of an exemplary slip-free mechanism;

FIGS. 7A and 8A are cross sectional and perspective views, respectively, of an exemplary tissue engagement section;

FIG. 9A is a cross sectional view of an exemplary suture and ferrules;

FIG. 10A is a cross sectional view of an exemplary needle and spherical member;

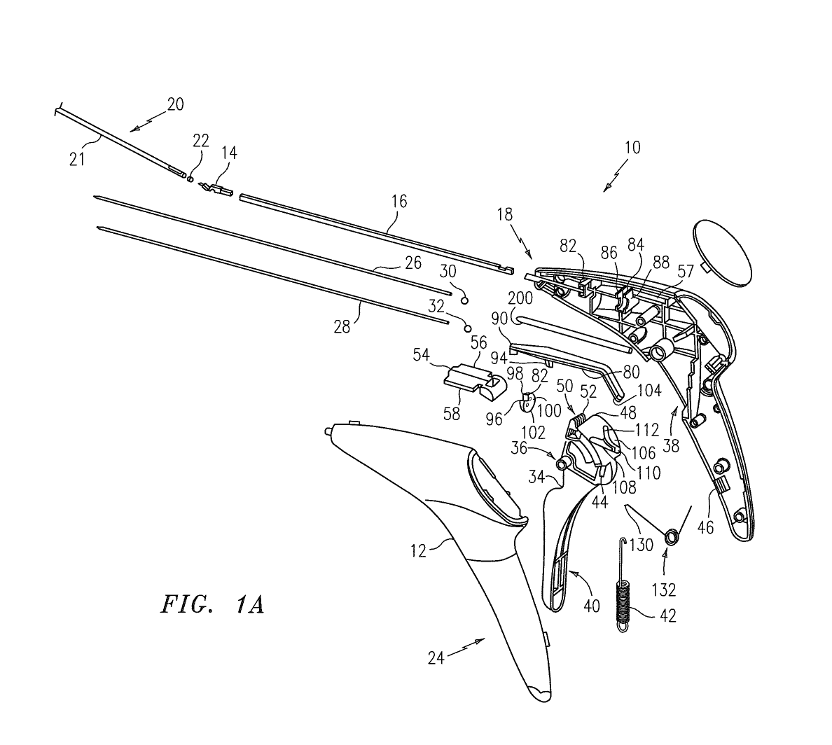

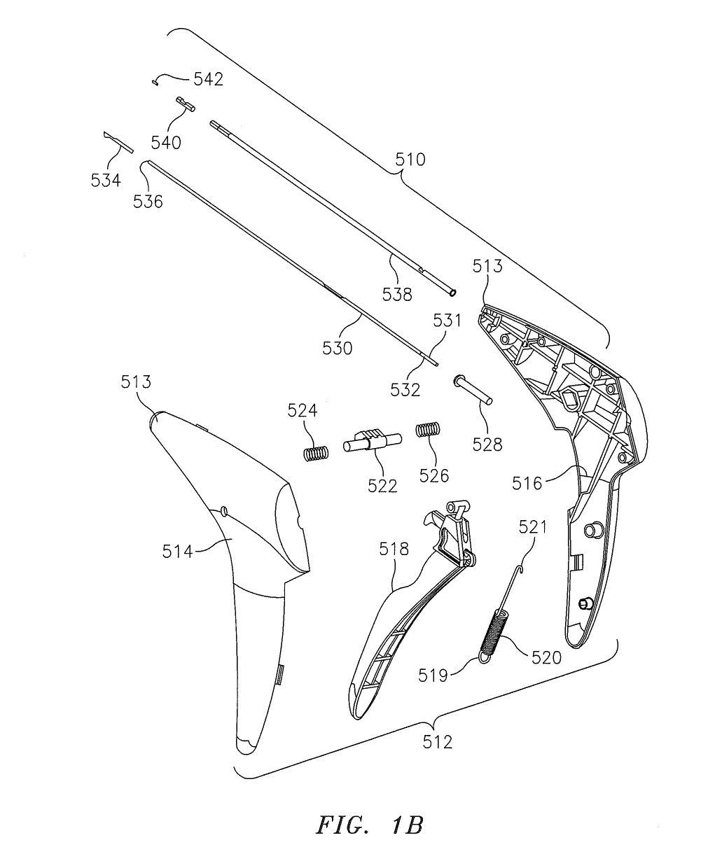

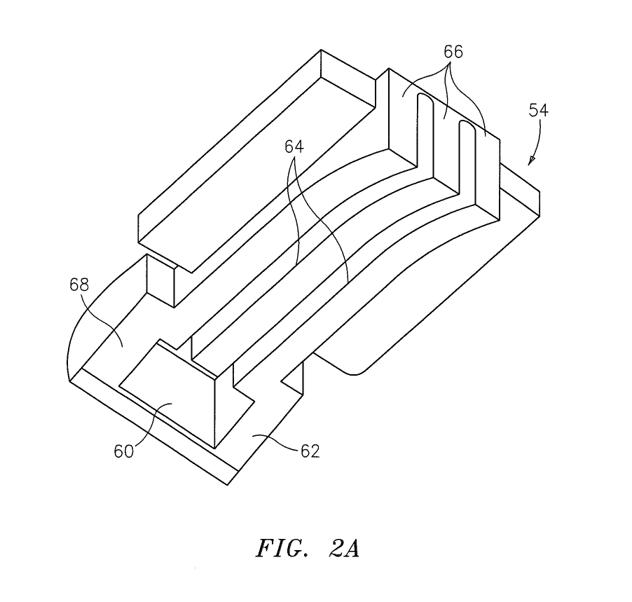

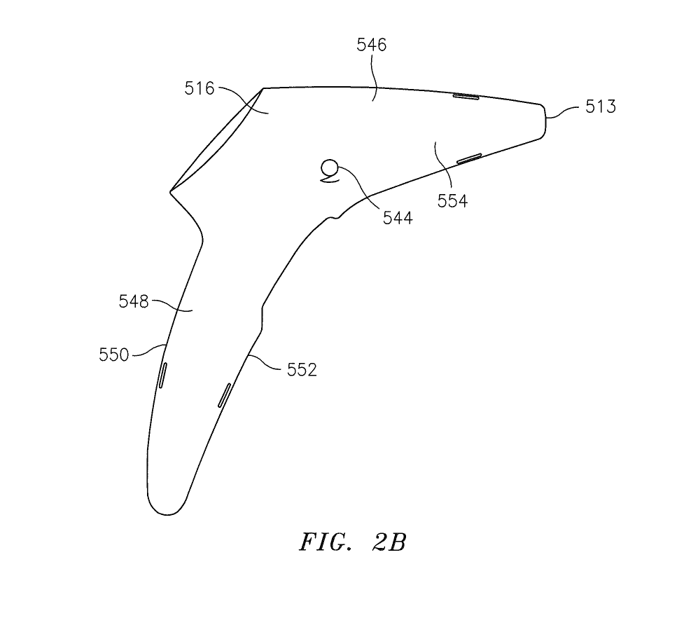

FIG. 1B shows an exploded perspective view of the cutting and crimping device;

FIG. 2B shows a side plan view of an exterior of the handle assembly for the cutting and crimping device of FIG. 1B;

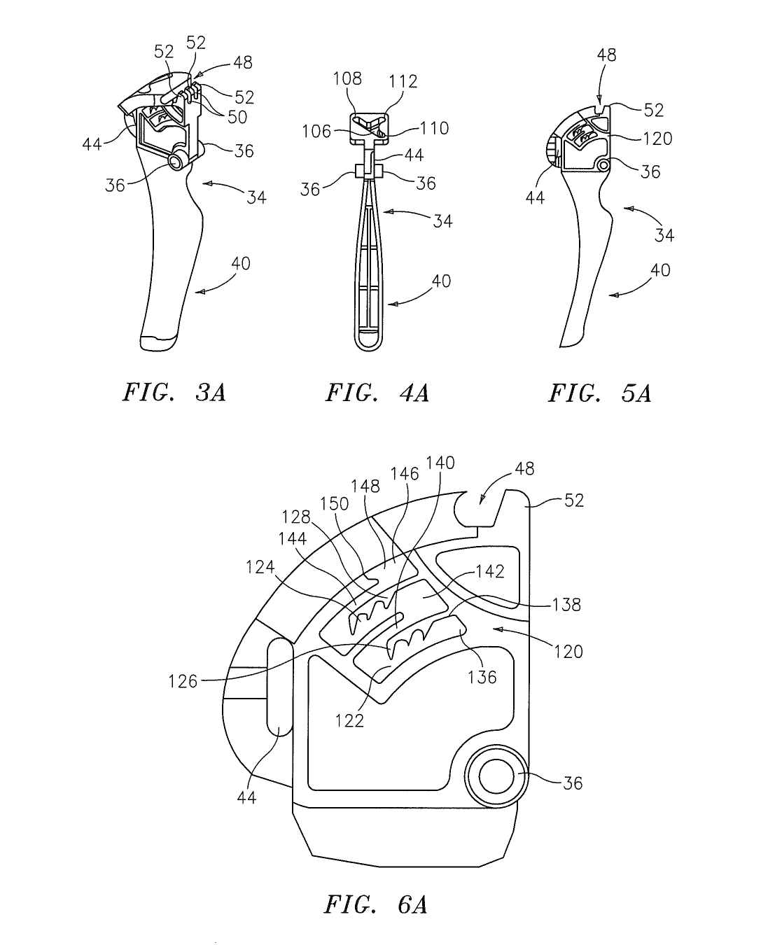

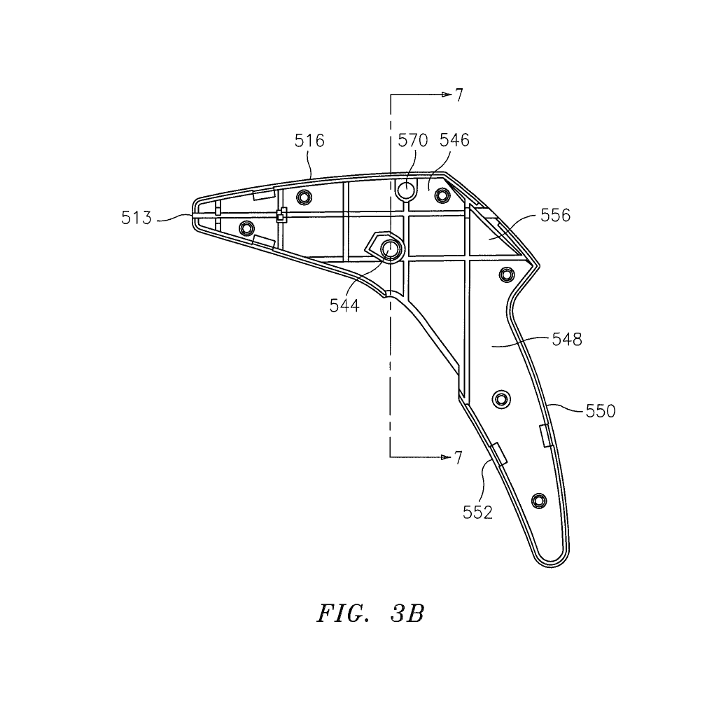

FIG. 3B shows an interior plan view of a side of the handle assembly for the cutting and crimping device of FIG. 1B;

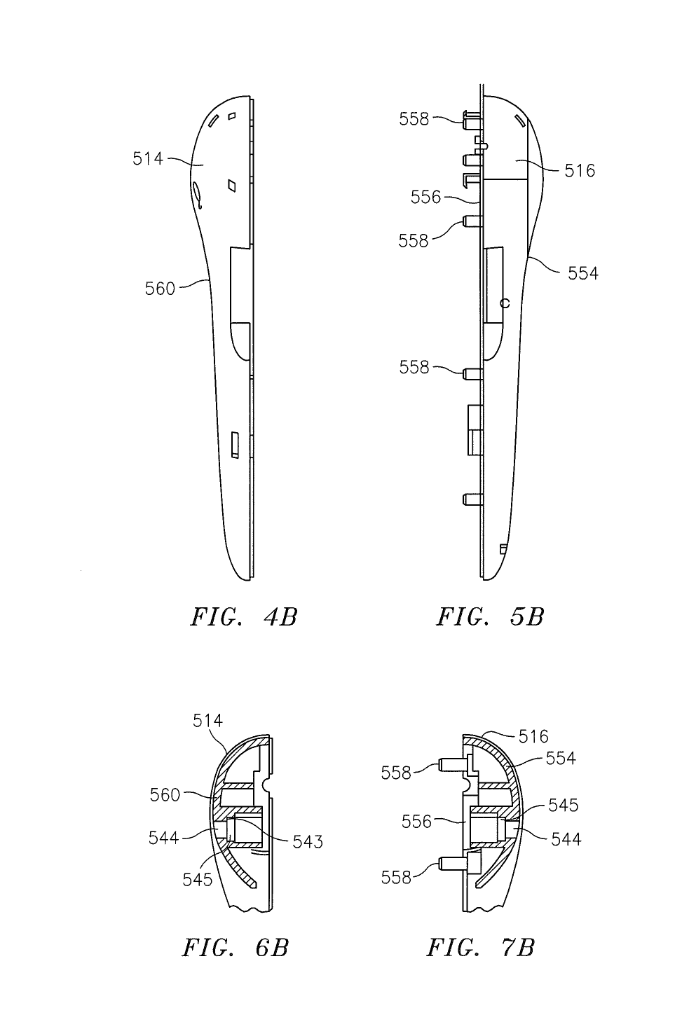

FIG. 4B shows a proximal plan view of a side of the handle assembly for the cutting and crimping device of FIG. 1B;

FIG. 5B shows a proximal plan view of another side of the handle assembly for the cutting and crimping device of FIG. 1B;

FIG. 6B shows a partial cross-sectional view of the side of FIG. 4B;

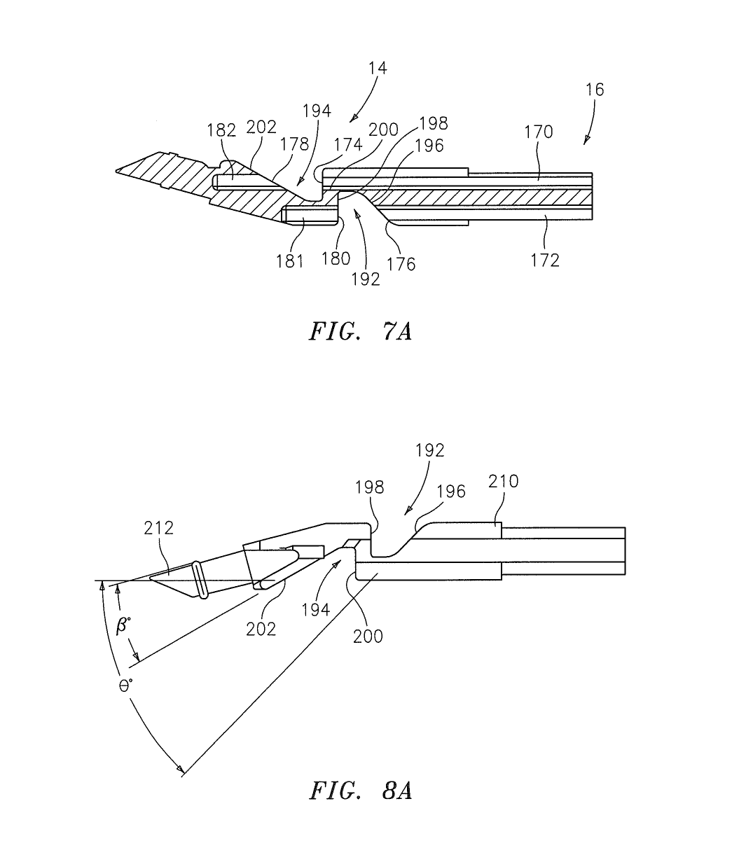

FIG. 7B shows a partial cross-sectional view of the side of FIG. 5B taken along line 7-7 of FIG. 3B;

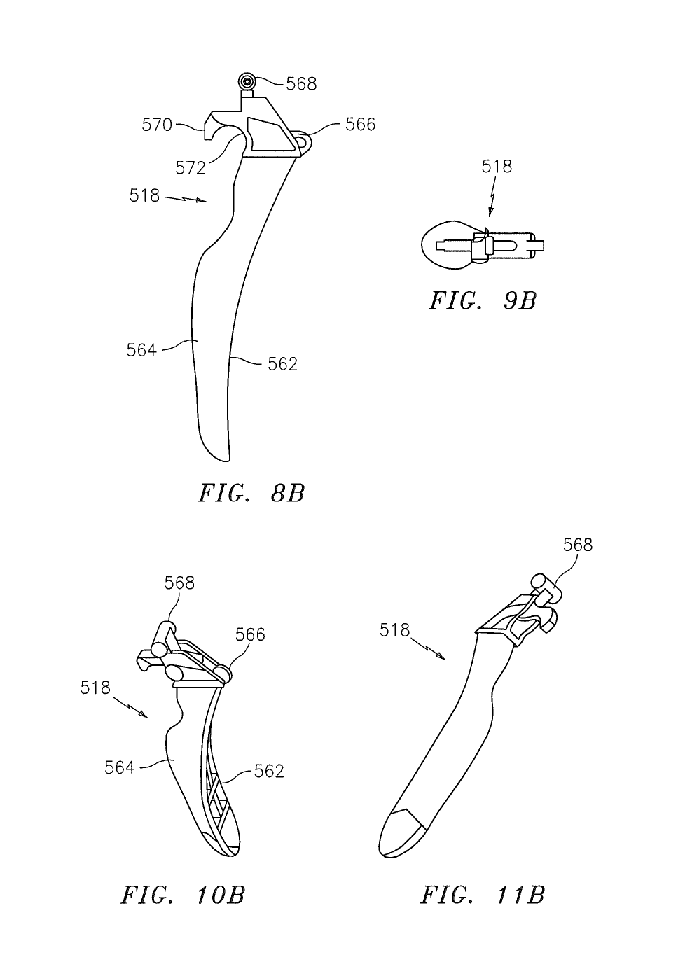

FIG. 8B shows a side plan view of the trigger of the handle assembly for the cutting and crimping device of FIG. 1B;

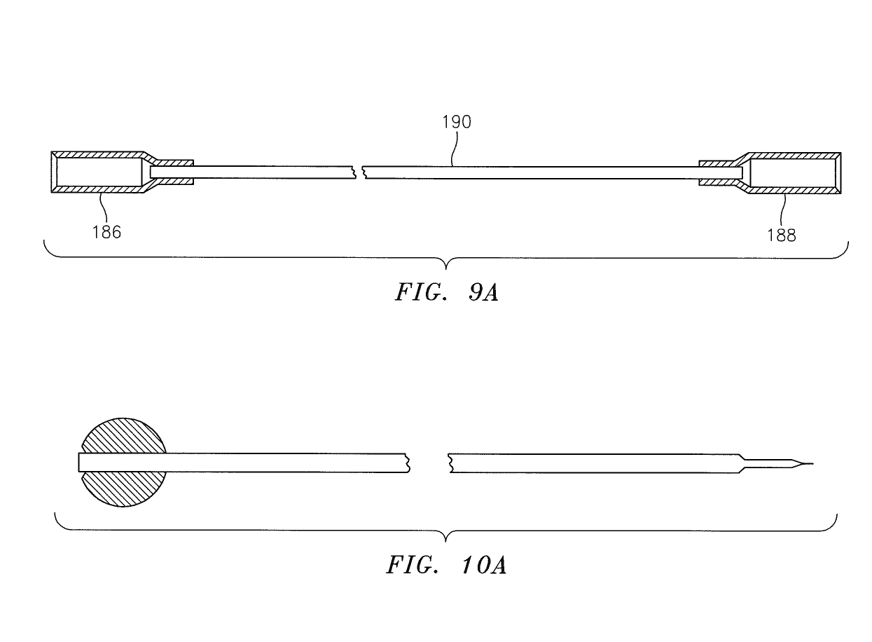

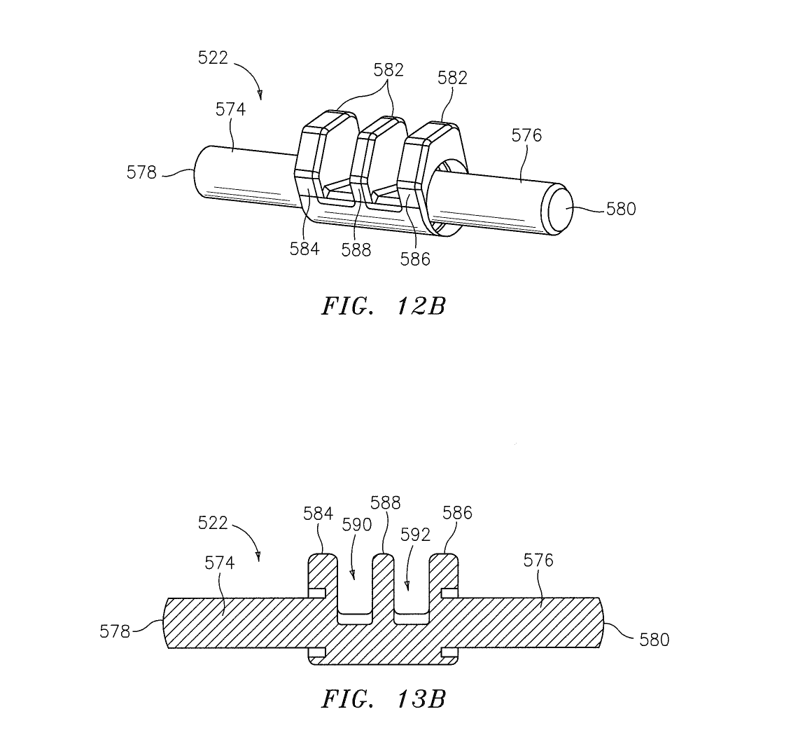

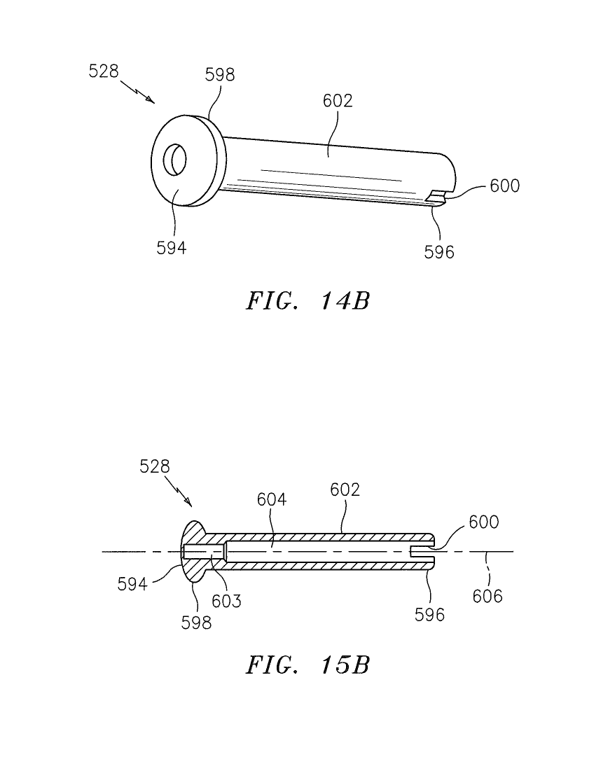

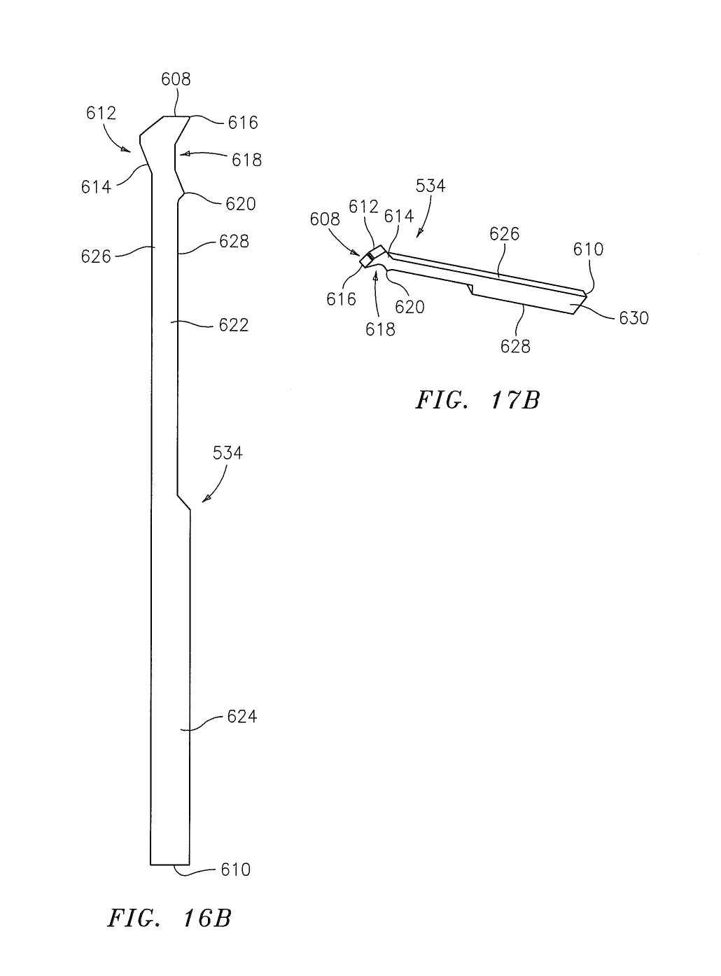

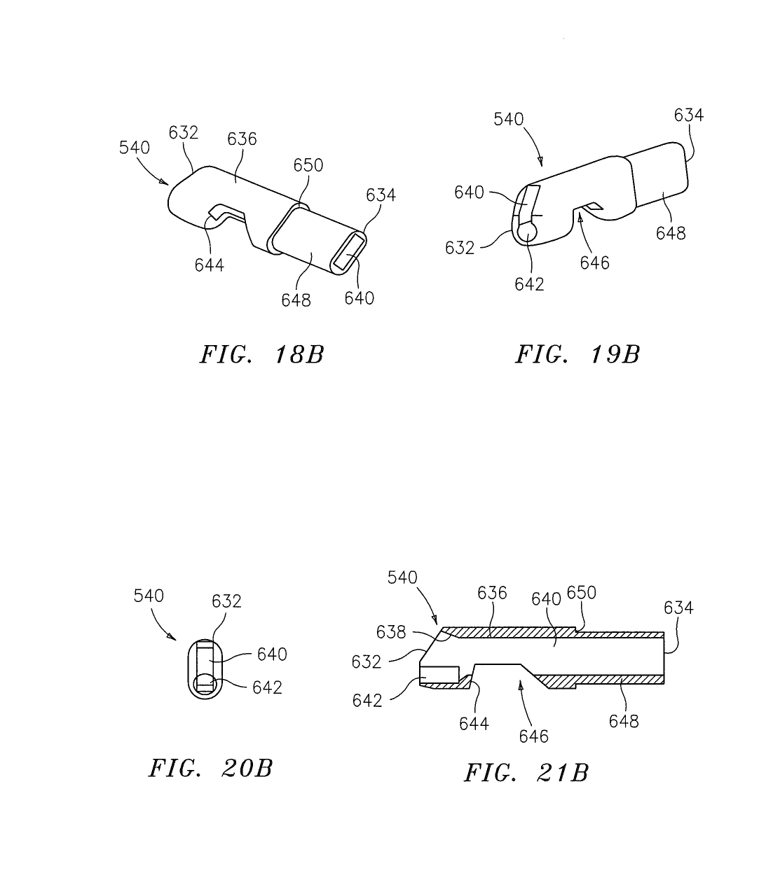

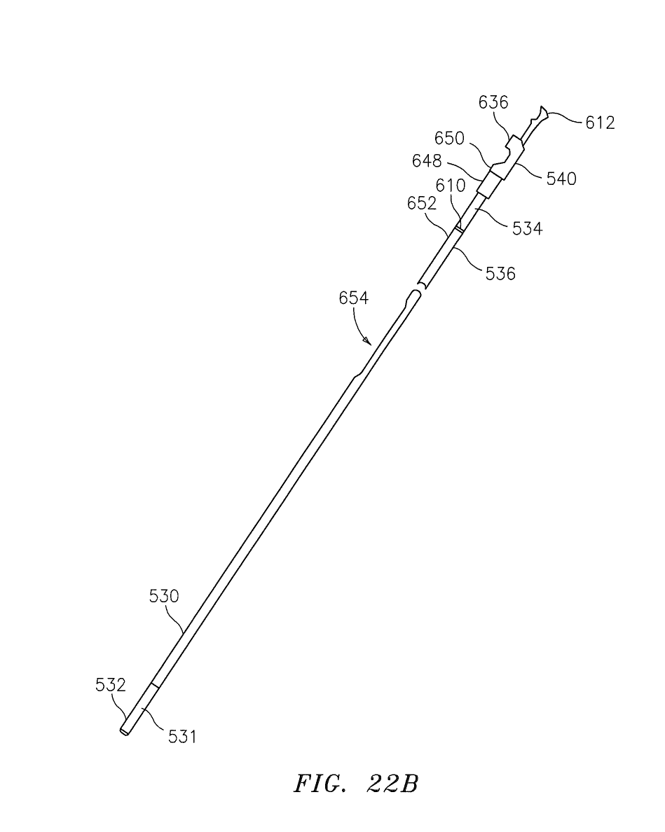

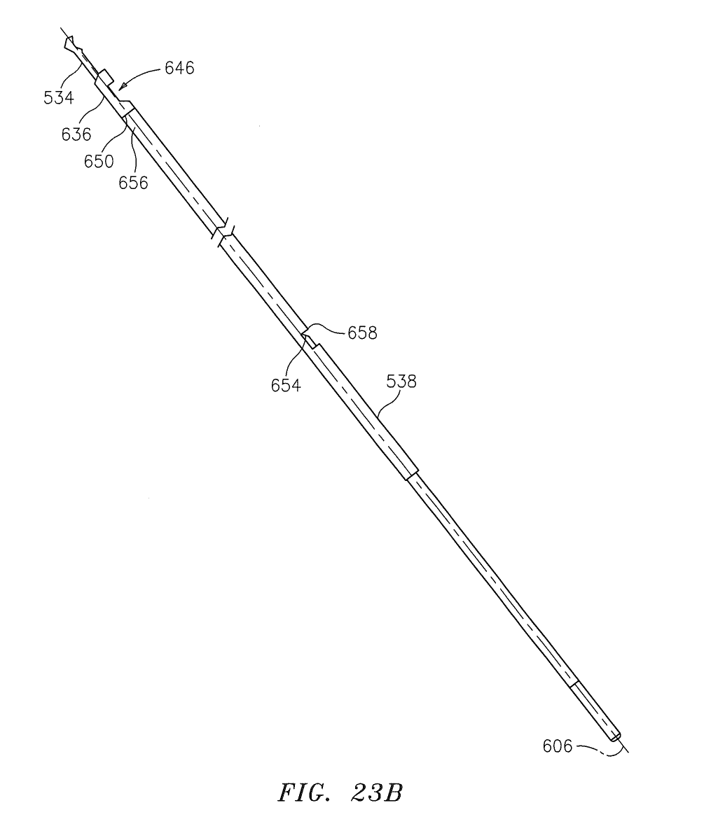

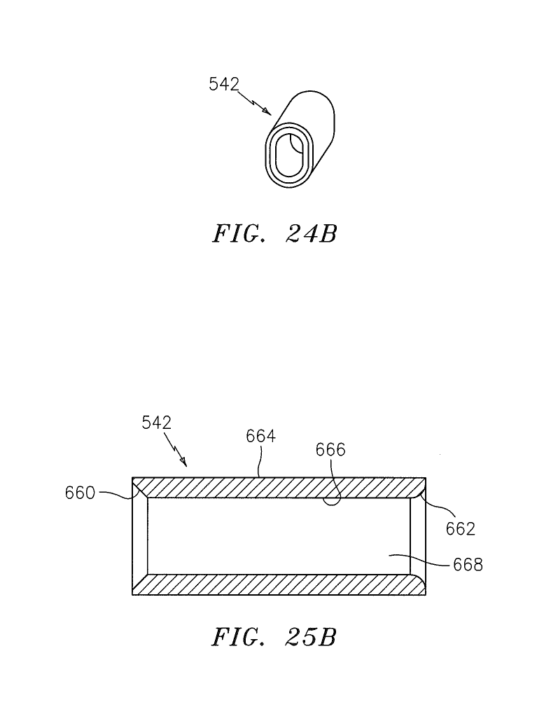

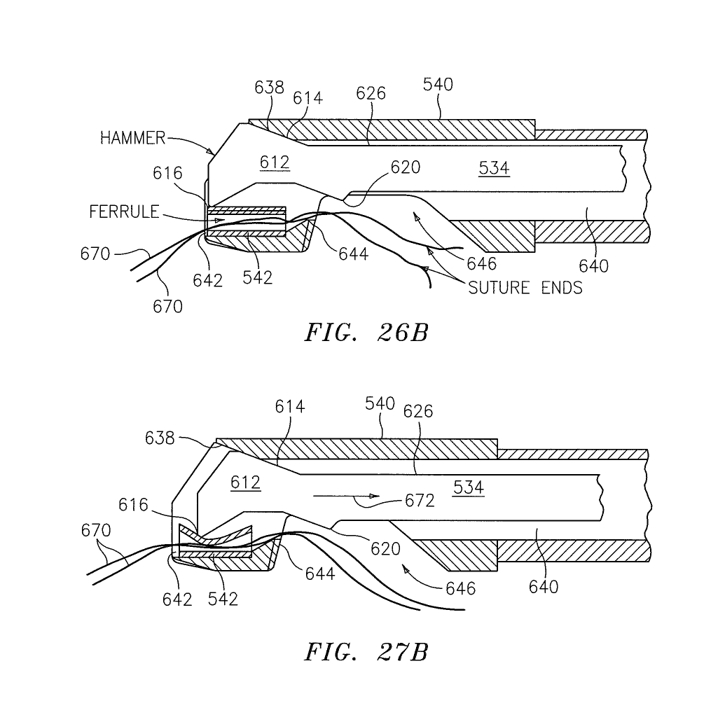

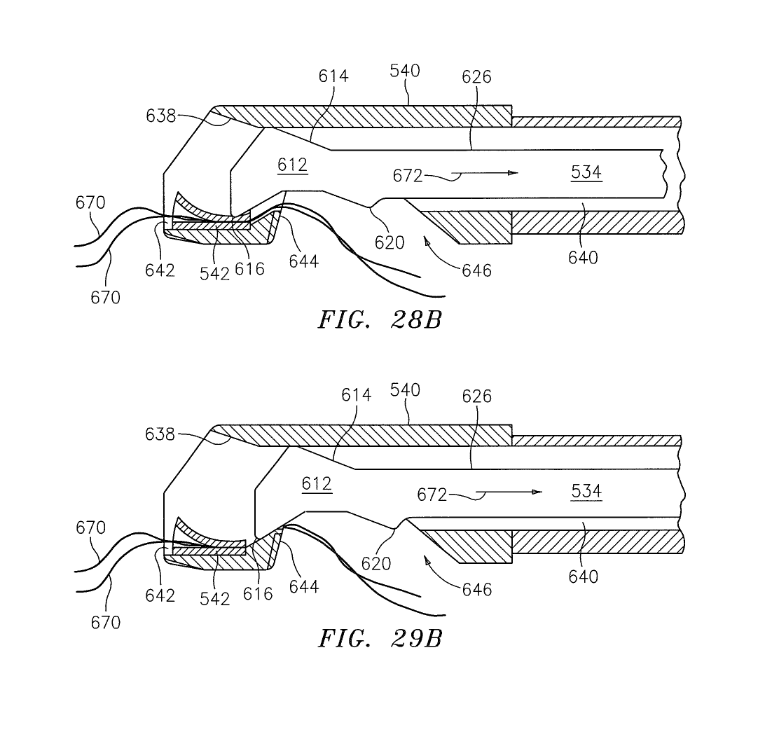

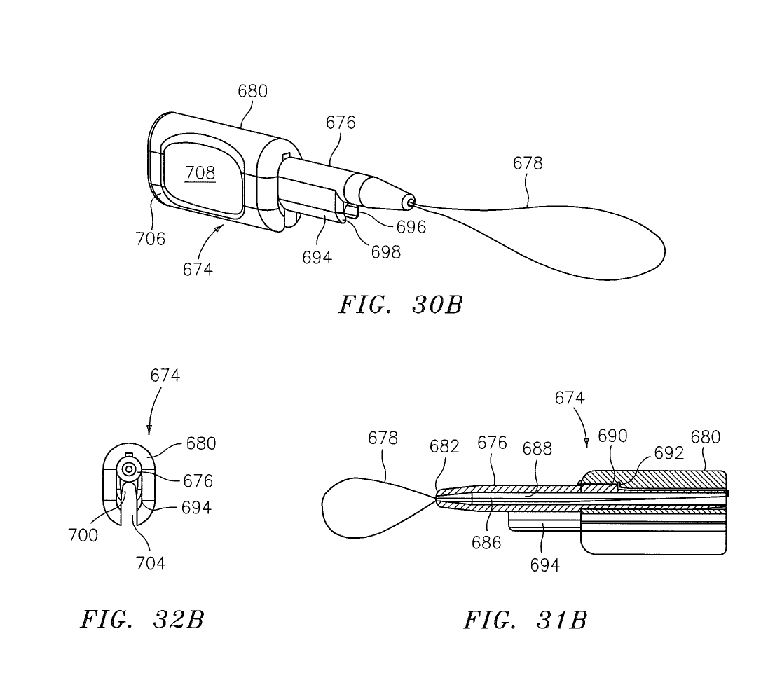

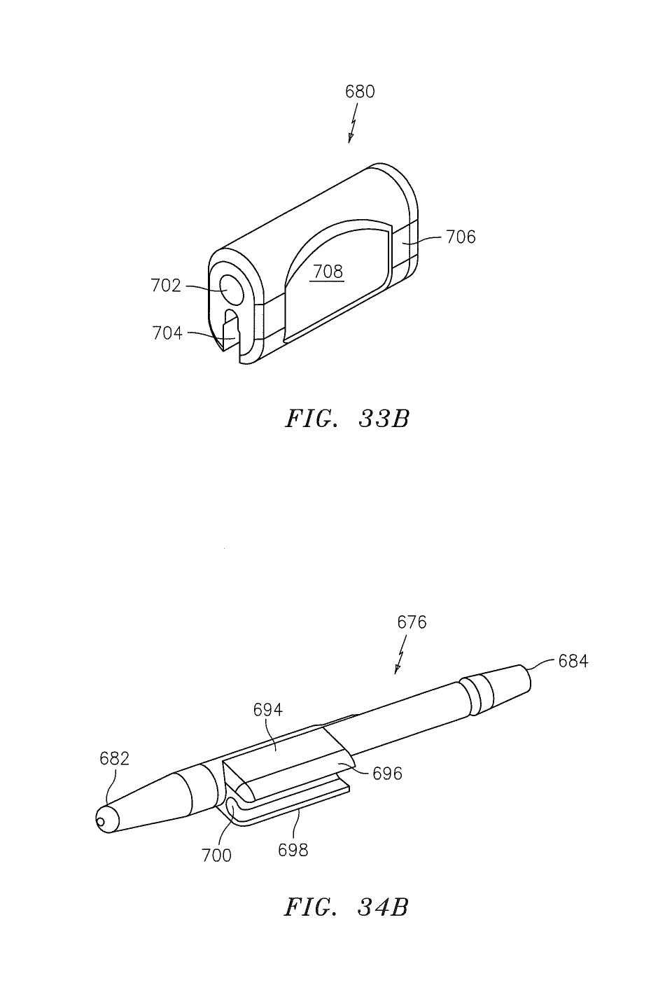

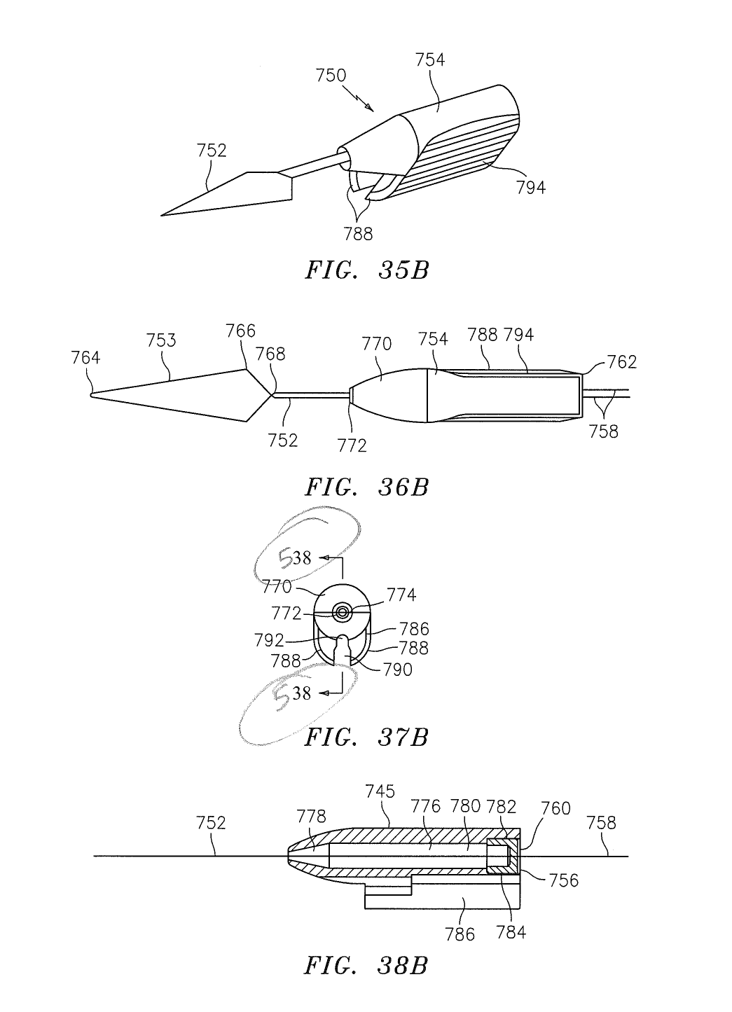

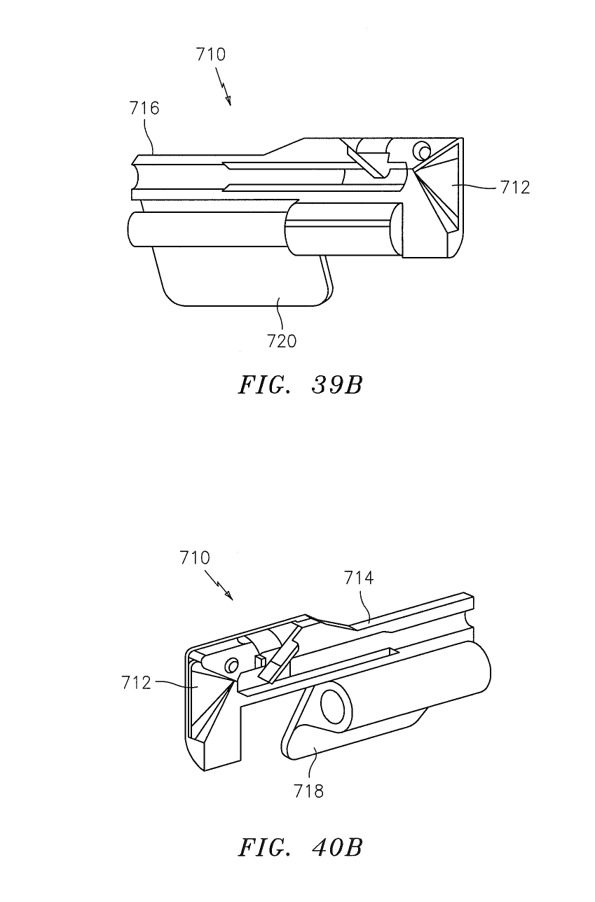

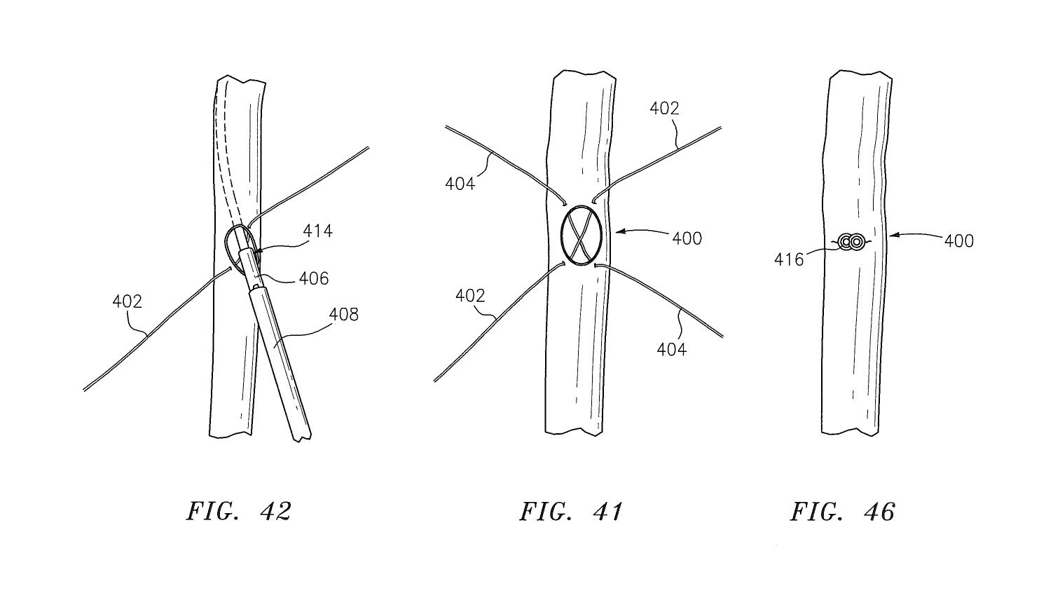

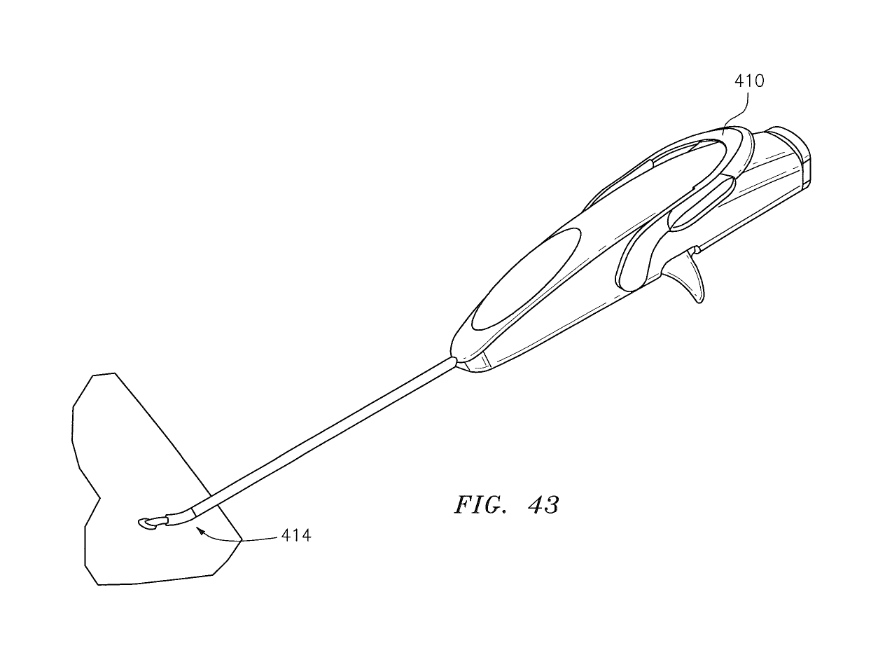

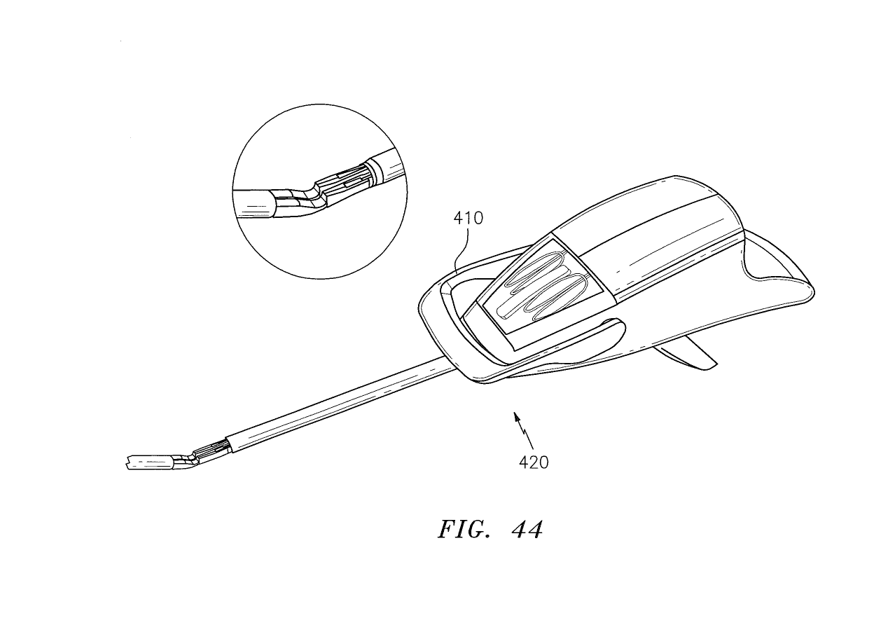

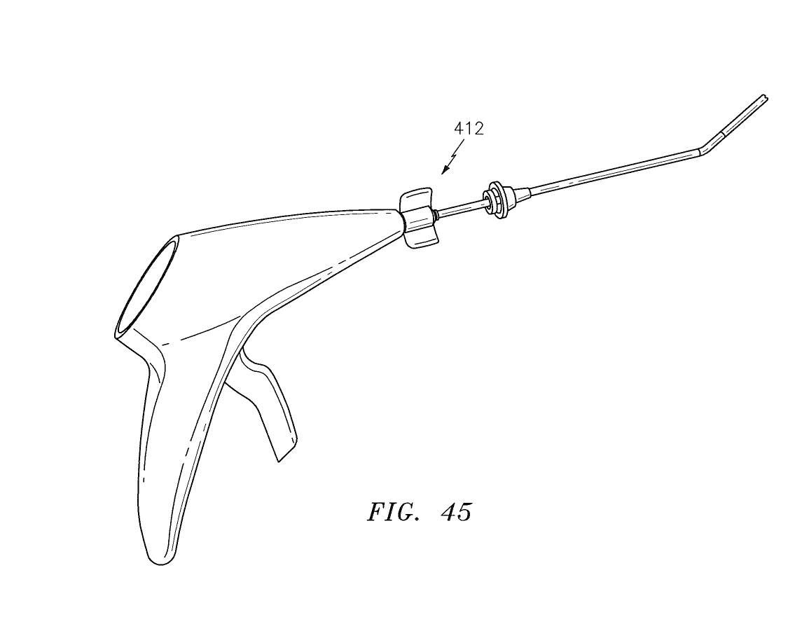

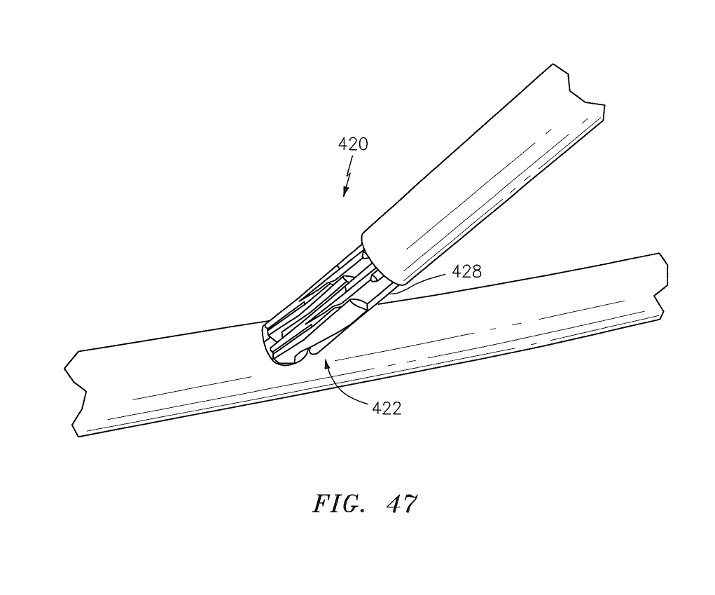

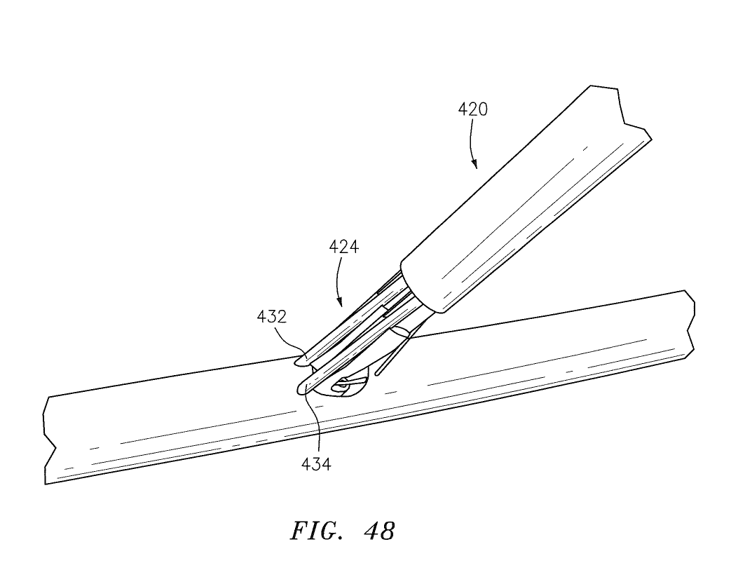

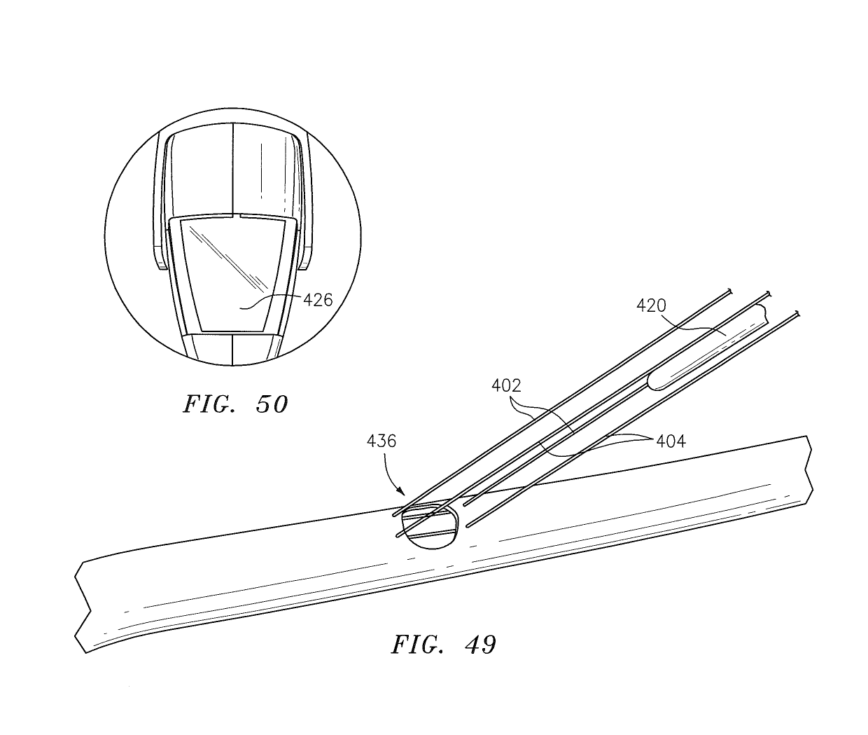

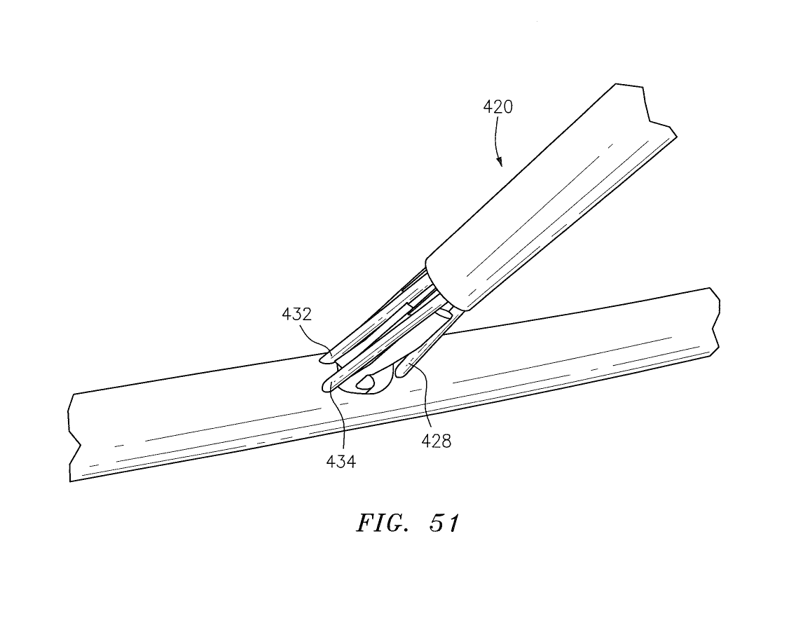

FIG. 9B shows a top plan view of the trigger of FIG. 8B;

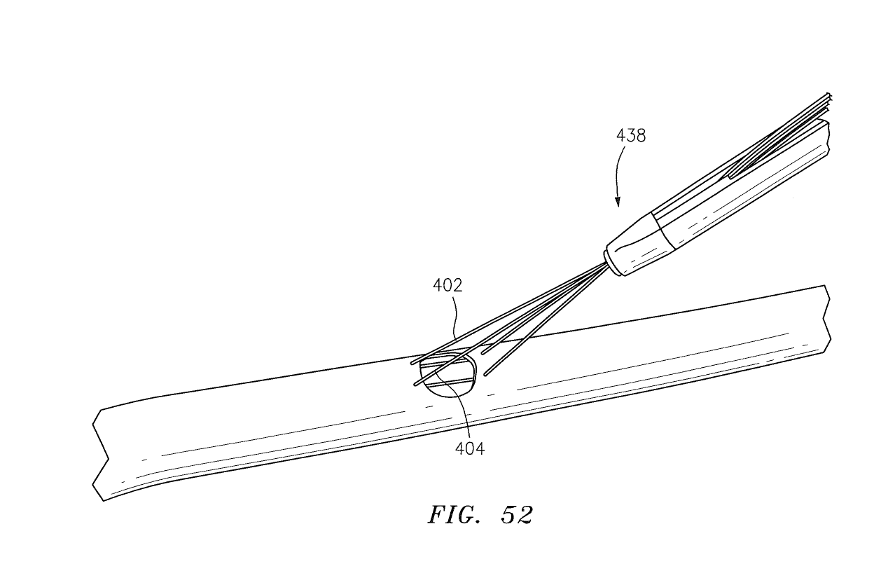

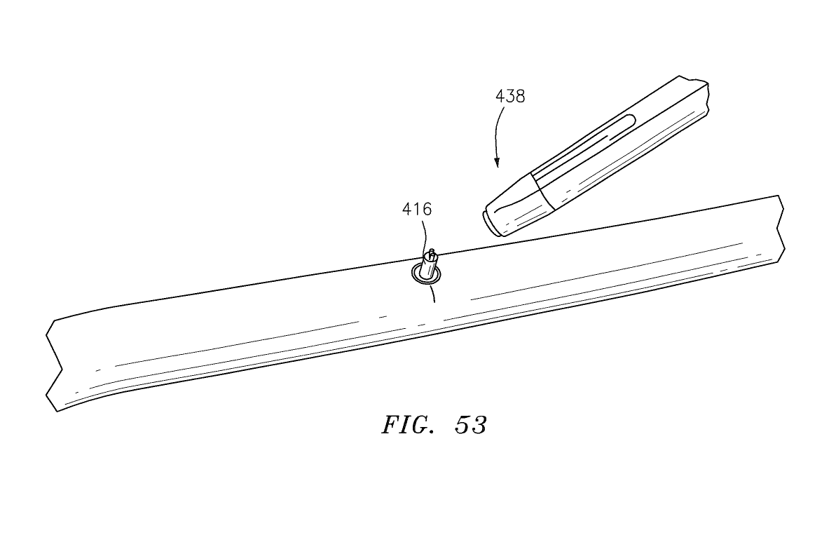

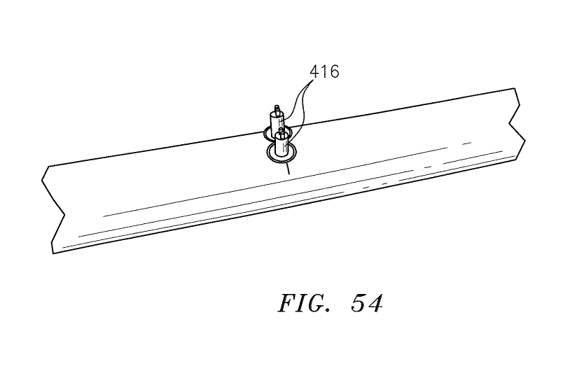

FIG. 10B shows proximal perspective view of the trigger of FIG. 8B;

FIG. 11B shows a distal perspective view of the trigger of FIG. 8B;

FIG. 12B shows a perspective view of the safety button of the handle assembly for the cutting and crimping device of FIG. 1B;

FIG. 13B shows a cross-sectional view of the safety button of FIG. 12B;

FIG. 14B shows a perspective view of the adjustment screw of the handle assembly for the cutting and crimping device of FIG. 1B;

FIG. 15B shows a cross-sectional view of the adjustment screw of FIG. 14B;

FIG. 16B shows a side plan view of the hammer element for the cutting and crimping device of FIG. 1B;

FIG. 17B shows a perspective view of the hammer element of FIG. 16B;

FIG. 18B shows a perspective view of the tip for the cutting and crimping device of FIG. 1B;

FIG. 19B shows another perspective view of the tip of FIG. 18B;

FIG. 20B shows a distal plan view of the tip of FIG. 18B;

FIG. 21B shows a cross-sectional interior view of the tip of FIG. 18B;

FIG. 22B shows a side plan view of the central rod, hammer element, and tip of the cutting and crimping device of FIG. 1B;

FIG. 23B shows a side plan view of the central rod, hammer element, tip, and tubular portion of the cutting and crimping device of FIG. 1B;

FIG. 24B shows a perspective view of a ferrule for use in the cutting and crimping device of FIG. 1B;

FIG. 25B shows a side cross-sectional view of the ferrule of FIG. 24B;

FIG. 26B shows a partial side cross-sectional view of the cutting and crimping device of FIG. 1B in an initial stage of securing suture material;

FIG. 27B shows a partial side cross-sectional view of the cutting and crimping device of FIG. 1B in an advanced stage of securing suture material;

FIG. 28B shows a partial side cross-sectional view of the cutting and crimping device of FIG. 1B in a further advanced stage of securing suture material;

FIG. 29B shows a partial side cross-sectional view of the cutting and crimping device of FIGURE lB in a final stage of securing suture material;

FIG. 30B shows a perspective view of one embodiment of a suture loading assembly;

FIG. 31B shows a side cross-sectional view of the suture loading assembly of FIG. 30B;

FIG. 32B shows a front plan view of the suture loading assembly of FIG. 30B;

FIG. 33B shows a side perspective view of a cap for use in the suture loading assembly of FIG. 30B;

FIG. 34B shows a side perspective view of a body for use in the suture loading assembly of FIG. 30B;

FIG. 35B shows a side perspective view of another embodiment of a suture loading assembly;

FIG. 36B shows a top plan view of the suture loading assembly of FIG. 35B;

FIG. 37B shows a front plan view of the suture loading assembly of FIG. 35B;

FIG. 38B shows a side cross-sectional view of the suture loading assembly of FIG. 35B taken along line 38-38 within FIG. 37B;

FIG. 39B shows a side perspective view of a half of another embodiment of a suture loading assembly; and,

FIG. 40B shows a side perspective view of another half of the suture loading assembly of FIG. 39B;

FIG. 41 illustrates a top elevation view of dual sutures across a wound;

FIG. 42 illustrates a top elevation view of a large-bore arterial wound receiving a second suturing device across a large bore arterial wound;

FIG. 43 is a perspective view of a suturing device with a retracted sheath;

FIG. 44 is a perspective view of an exemplary large bore plural suture application device;

FIG. 45 is a side elevation view of an exemplary device with a wingnut sheath manipulator;

FIG. 46 is a top elevation view of an approximated wound using one ferrule and dual sutures;

FIG. 47 illustrates an exemplary first deployment of plural needles at a lower, first tissue receiving gap;

FIG. 48 illustrates deployment of third and fourth needles across a second, upper tissue receiving gap;

FIG. 49 illustrates removal of the exemplary plural suture applying device with plural sutures approximating the large bore wound;

FIG. 50 illustrates successful capture each suture via a vacant suture window;

FIG. 51 shows an alternate embodiment facilitating deployment of dual needles across each tissue gap simultaneously;

FIG. 52 illustrates an exemplary embodiment, wherein plural sutures are loaded through a cinching device that is configured to receive the plural sutures;

FIG. 53 illustrates crimping of the plural sutures inside a ferrule, wherein the ferrule experiences crimping forces from different sides; and

FIG. 54 illustrates an alternate embodiment using plural ferrules with plural crimping to capture separate sutures and approximate a large bore wound.

### DETAILED DESCRIPTION

The above described and other disadvantages of the prior art are overcome and alleviated by the present wound suturing device, which comprises a system for wound suturing and crimping and cutting of wound sutures.

### DESCRIPTIONS OF EXEMPLARY SUTURING DEVICES

An apparatus for applying a suture to body tissue is illustrated in FIG. 1A and is designated generally by reference numeral 10. Note that the terms “first” and “second” as used herein are for the reader's convenience and should not be interpreted as necessarily denoting the order in which the components are actuated.

Referring to FIG. 1A, an exemplary wound suturing apparatus 10 is shown having a housing 12, a tissue engaging portion 14, a shaft 16 extending from an opening 18 in the housing to the tissue engaging section 14, and a flexible guide tube 20 coupled at 22 to the tissue engaging section 14. The housing 12 has a body shaped like a pistol having a handle portion 24, and is illustrated in the exemplary embodiment as a two-piece construction of molded plastic. The apparatus 10 includes a pair of needles 26 and 28, which extend from housing 12 through the shaft 16 into the tissue engaging section 14. Each needle 26 and 28 has a non-tissue engaging end in the housing having a spherical member 30 and 32, such as a ball or bearing, respectively, attached thereto. Both needles 26 and 28 and spherical members 30 and 32 may be a made of metal, such as surgical stainless steel. The spherical members 30 and 32 may have a bore into which the non-tissue engaging ends of the needles 26 and 28, respectively, extend and joined thereto, such as by welding.

The apparatus 10 includes an actuator member 34 having two pins 36 extending into holes in the sides of housing 12 upon which the actuator member is pivotally mounted in the housing. Actuator member 34 has a portion that extends through an opening 38 in housing 12 to provide a trigger 40. A coil spring 42 is provided which hooks at one end in a notch 44 of actuator member 34 and is wound at the other end around a pin 46 located in holes in the sides of housing 12, such that the actuator member 34 is spring biased to retain trigger 40 normally in a forward position, as shown for example in FIG. 1A. A notch 48 is provided in the actuator member 34 which is shaped to receive one of the non-engaging ends of needles 26 or 28, i.e., spherical members 30 or 32, to be driven forward by the actuator member 34 by a user pulling the trigger portion 40 of actuator member 34 towards handle portion 24. Two grooves 50 are provided by three fingers 52 into which portions of the needles 26 or 28 proximate to the spherical members 30 or 32, respectively, may lie.

A retainer member 54 is fixed in housing 12 by two flanges 56, 58 above the actuator member 34 within mating surfaces 57. As best shown in FIG. 2A, the retainer member 54 has a chamber 60 having a lower opening 62 and two grooves 64 formed by fingers 66 which allow the spherical members 30 or 32 of needles 26 or 28, respectively, to be received in chamber 60 to restrict movement of the needle when held therein. The lower surface 68 of retainer member 54 is curved and faces correspondingly shaped fingers 52 of actuator member 34, such that the actuator member 34 is slidable along lower surface 68 responsive to a user pulling and releasing trigger 34.

Referring now to FIGS. 3A-6A, an exemplary actuator mechanism 34 is shown in detail by the various perspective views. As described above, the actuator mechanism 34 includes a trigger portion 40, pins 36, a notch 44 for receipt of a coil spring 42, a notch 48 shaped to receive one of the non-engaging ends of needles 26 or 28, and grooves 50 provided by three fingers 52 into which portions of the needles 26 or 28 proximate to the spherical members 30 or 32, respectively, may lie.

Referring again to FIG. 1A, a needle selection mechanism is provided including a selector lever (or arm) 80, which is rotationally coupled with a cam member 82. The cam member 30 is supported by an adapter 84 in housing 12. The cam member 82 is mounted in housing 12 by two flanges 86, 88. The selector lever 80 is pivotally mounted by a pin 90 extending downwards from a distal end portion of the selector lever into a notch 92 in the housing 12. The selector lever 80 has a downwardly protruding member 94 which is received in a notch 96 of cam member 82 to rotate cam member 82 in a pocket between flanges 86 and 88 as the selector lever 90 is moved left or right. The cam member 82 has a tapered surface 98 to facilitate its rotation in pocket and two tapered apertures 100 and 102 through which needles 26 and 28 respectively extend. The selector lever 80 further includes a proximal pin 104 configured to engage a slot, shown generally at 106 on an upper portion of the actuator member 34.

In an initial configuration, the proximal pin 104 is positioned in a left lobe 108 of slot 106. During an initial actuation of the trigger portion 40, the proximal pin 104 of the selector mechanism 80 travels to trough 110 of the slot 106. During this actuation, the orientation of the cam member 82 is such that the needle 28 is in an engaged position within the grooves 50, 48 of the actuator member 34 while the needle 26 is disengaged from the grooves 50, 48. The lobes 108, 112 and trough 110 of the slot 106 is configured such that release of the actuated trigger portion 40 causes the proximal pin 104 to travel into the right lobe 112 of the slot 106. This causes the proximal portion of the selector mechanism 80 to shift to the right, and at the same time, causes the cam member 82 to rotate in the same direction. The cam member 82 urges the needle 28 out of the grooves 50, 48 in the actuator mechanism 34 and simultaneously urges the needle 26 into the grooves 50, 48 in the actuator mechanism 34. Thus, a second actuation of the trigger portion 40 urges needle 26 in a distal direction but does not actuate the needle 28.

Referring now to FIGS. 5A and 6A, the actuator mechanism 34 is provided with a slip-free mechanism, shown generally at 120. The slip-free mechanism 120 generally comprises dual ratchet tracks 122, 124, each with a plurality of ratchet teeth 126, 128. The ratchet tracks and teeth are configured to engage a traveling pin portion 130 of spring 132 (shown in FIG. 1A). Referring to FIG. 6A, the traveling pin portion 130 resides initially at a distal region 136 of ratchet track 122 and is biased against an upper wall 138 of the ratchet track 122. During initial actuation of the trigger portion 40 of the actuation member 34, the traveling pin portion 130 traverses the ratchet teeth 126. After a full actuation of the trigger portion 40, the traveling pin portion 130 moves into a first return channel 140. Release of the trigger portion 40 positions the traveling pin portion 130 at a distal region 142 of the second ratchet track 124. During a second actuation of the trigger portion 40, the traveling pin portion 130 traverses the ratchet teeth 128. After a full second actuation of the trigger portion, the traveling pin portion 130 moves into a second return channel 144. Release of the trigger portion 40 positions the traveling pin portion 130 at a distal region 146 of a lockout track 148. The traveling pin portion 130 is thereafter prevented from further movement by lockout stop 150.

Thus, the slip-free mechanism 120 and the traveling pin portion 132 of the spring 130 allow for only two actuations of the actuator mechanism 34. At the same time, the ratchet teeth 126 and 128 within the ratchet paths 122 and 124 prevent partial actuation of the actuator mechanism 34 and thus, partial deployment of the needles 26, 28.

Referring to FIG. 7A, to orient the needles 26 and 28 within the tissue engagement section 14, the two needles 26 and 28 are configured to travel through generally parallel tracks 170, 172 in an x-z plane as they exit the shaft 16 and cross the tissue engaging section 14. To maximize the separation of the needles 26 and 28, the shaft 16 is oval in cross-section, having a major axis of the oval (though the cross-section may be circular or any convenient shape) for at least a substantial portion of the shaft as it extends to shaft end near the tissue engaging section 14. The tissue engaging section 14 of the tissue suturing apparatus 12 further includes a first opening 174, a second opening 176, and third 178 and fourth 180 openings providing access to distal channels 182 and 184, which are each capable of holding a needle capturing portion 186 and 188, respectively (see FIG. 9A), received through openings 178 and 180, respectively. Needle capturing portions 58a and 60a are referred to herein as ferrules, such as described, for example, in U.S. Pat. Nos. 5,431,666 and 5,766,183, but may be any means by which a suture may be captured at the tip of a needle. The ferrules 186 and 188 each have an opening to an interior cavity shaped to enable the ferrule to frictionally engage the end of the needles 26 and 28, respectively, when received in the interior cavity. Each ferrule may be made of metal or plastic and may be oval in cross-section such that they can frictionally engage the tip of a needle. The ferrules 186 and 188 are each connected to one end of the two ends of a length of suture material or thread 190 extending through a suture tube or channel (not shown) positioned either in the elongated body 16 or in the flexible member 20.

In another embodiment one or more of the ferrules 186, 188 includes an interior cavity with an angled cross section providing multiple lines of interference. In another embodiment, the interior cavities of the ferrules 186, 188 have triangular cross sections, providing three lines of interference during engagement of the ferrule interiors with the distal tips of the needles. In another embodiment, the interior cavities of the ferrules 186, 188 have square cross-sections, providing four lines of interference during engagement of the ferrule interiors with the distal tips of the needles.

With reference to FIGS. 7A and 8A, the tissue engaging section 14 has a first gap 192 and a second gap 194 in which the first gap 192 is along the lower side of section 14 and the second gap 194 is along the opposite upper side of section 14 and forward with respect to the first gap along the length of the section 14 in a direction distal from housing 12. The first gap 192 has two opposing surfaces 196 and 198 into which one side of a wound can be received, where opening 176 is located along surface 196 and opening 180 to ferrule holder 184 is located along surface 198 facing opening 176. Similarly, the second gap 194 has two opposing surfaces 200 and 202 into which the other side of the wound can be received, where opening 174 is located along surface 200 and the opening 178 to ferrule holder 182 is located along surface 202 and faces opening 174. Each gap 192 and 194 is shaped to have a depth to facilitate the placement of the edge of a wound therein. Surface 198, which is the distal face of the first gap 192, and surface 202, which is the proximal face of the second gap 194 both serve as stop surfaces for the tissue engaging section 14. Such stop surfaces 198, 200 assist in the placement of the tissue engaging section 14 relative to the wound as will be described further below. End portions 210 and 212 of the tissue engaging section are angled with respect to each other as shown in FIG. 8A to facilitate placement of end 212 with guide section 20 through a sheath (or cannula) and the puncture wound to maximize blood vessel engagement. The two ferrules 186 and 188 and suture material 190 may be located in apparatus 10 during manufacture.

With reference to FIG. 8A, in one embodiment, stop surfaces 198 and 200 are squared at a middle portion thereof to provide good tactile feel to the surgeon of the stop points. In another embodiment, the stop surfaces may have an angle with regard to the longitudinal axis of the first end portion 210 of the tissue engaging section 14 of between about 85 and 95 degrees. In another embodiment, the stop surfaces have an angle of about 90 degrees.

Referring still to FIG. 8A, in one embodiment, wall 196 of the first gap 192 has an angle theta of between about 40 and 50 degrees with regard to the longitudinal axis of the first end portion 210 of the tissue engaging section 14. In another embodiment, wall 196 has an angle theta of about 45 degrees.

Referring still to FIG. 8A, in one embodiment, wall 202 of the first gap 194 has an angle beta of between about 25 and 35 degrees with regard to the longitudinal axis of the first end portion 210 of the tissue engaging section 14. In another embodiment, wall 202 has an angle beta of about 30 degrees.

Thus, by the above described exemplary ranges of the geometry of the tissue engaging section 14, an aggressive tissue contacting surface is described to facilitate bite of tissue, particularly for wound suturing devices having small sizes, where positive tissue capture and stop indication is particularly advantageous. In one embodiment, the tissue engaging section 14 has a size between about 6 and 8 French. In another embodiment, the tissue engaging section 14 has a size of about 7 French.

The tissue engagement section 14 may be made of metal, such as stainless steel, or other rigid biocompatible material. For example, the tissue engagement section may be made of two pieces of shaped metal having bores providing the desired openings, channels, and receptacles, joined together down the middle along section by welding or heat shrinking of heat shrinkable tubing connecting the two pieces. The components in the housing 12, such as the actuator member 34, selector lever 80, and needle retainer 220, may be made of molded plastic.

A guide section 20 is attached to end 212 (FIG. 8A) of the tissue engaging section 14. As shown best in FIG. 1A, the guide section 20 has a flexible tube 21 having an opening (not shown) through which a guide wire may be received. The tube 20 may be made of a biocompatible plastic, like heat shrink tubing, and the ramp may be made of plastic or metal, which is attached or joined within tube 20.

### DESCRIPTIONS OF EXEMPLARY CUTTING AND CRIMPING DEVICES

Referring to FIG. 1B, a crimping and cutting device 510 is shown for applying a ferrule around suture material after it has been applied to body tissue by a suturing apparatus, or by any other suturing procedure in which suture material is used, in order to secure the suture.

In general, device 510 preferably includes a handle assembly 512. The handle assembly may include first and second sides 514, 516 and a trigger member 518 with an associated spring 520 for the trigger return. The handle assembly 512 further preferably includes a safety button 522 which is centrally biased by springs 524, 526 and which must be depressed before trigger actuation will be permitted. Also preferably within the handle assembly 512 is an adjustment screw 528 which facilitates ferrule loading by the manufacturer. The adjustment screw is connected to a proximal end 532 of a central rod 530, which extends from a distal end 513 of the handle assembly 512. A hammer element 534 is connected to the distal end 536 of the central rod 530. Surrounding the central rod 530 is a tubular portion 538 which also extends from a distal end 513 of the handle assembly 512. A tip 540 is secured to the proximal end of the tubular portion 538. Positioned within the tip 540 is a ferrule 542.

An exterior 554 of side 516 of handle assembly 512 is shown in FIG. 2B and FIG. 3B shows an interior 556 of side 516, similar to that shown in FIG. 1B. The handle assembly 512 includes a distal end 513 from which the central rod 530 and tubular portion 538 extend. The side 516 includes an opening 544 through which a portion of the safety button 522 extends, as will be further described. The side 516, as with the side 514, preferably includes a body portion 546 and a grasping portion 548. The body portion 546 houses the safety button 522 and the adjustment screw 528 and includes the connections for the central rod 530, tubular portion 538, and trigger 518. The grasping portion 548, on the hand, preferably includes an ergonomically shaped grip for an operator. The grasping portion 548 includes an outward surface 550 and an inward surface 552. The inward surface 552 faces an inner surface of the trigger 518.

As shown in FIGS. 4B and 5B, the interior 556 of side 516 is shown to include protrusions 558 which mate with corresponding shaped recesses (not shown) within the interior of side 514 during manufacture. Alternatively, the protrusions 558 could be located on side 514 with recesses within interior 556, or some of the protrusions 558 could be located on both sides 514 and 516 with corresponding recesses oppositely positioned on sides 514 and 516. When assembled, an exterior 560 of the side 514 and the exterior 554 of side 516 preferably combine to form a smooth outer surface of the handle assembly 512 for gripping by the operator.

Turning now to FIGS. 6B and 7B, a cross-section of the sides 514 and 516 is shown. Specifically, a cross-section of side 516 taken along line 7-7 within FIG. 3B is shown in FIG. 7B. A cross-section of side 514 taken along the same location, that is, through the opening 544 for safety button 522, is shown in FIG. 6B. The opening 544 extends from the exterior 554 of side 516 to the exterior 560 of side 514. Thus, the safety button, as will be further described below, is accessible from either side 514 or 516 of the handle assembly 512.

Turning now to FIGS. 8B-11B, the trigger 518 is shown to include an inner surface 562 which, when assembled within the handle assembly 512, faces the inward surface 552 of the sides 514 and 516. Outer surface 564 is preferably smooth for grasping by an operator. The trigger 518 may include spring receiving member 566 for receiving a hook 521 (as shown in FIG. 1B) of the spring 520. As shown, the spring receiving member 566 is an opening, although other shapes, such as a hook shape would be within the scope of this invention. With the hook 521 of the spring 520 engaged within the spring receiving member 566 of the trigger 518 and a securing member 519 (FIG. 1B) of spring 520 securing the spring 520 to a protrusion 558 on side 516, the trigger 518 must expand the spring 520 when the inner surface 562 of the trigger 518 is compressed towards the inward surface 552 of the sides 514, 516. The spring 520 thus biases the trigger 518 in an “open” or “unsqueezed” configuration. That is, after the trigger 518 is squeezed, the spring 520 will return the trigger 518 to its original position when pressure on the trigger 518 is removed.

The trigger 518 further preferably includes a pivot rod 568 for pivotally securing the trigger 518 within the handle assembly 512, such as within opening 570 within interior 556 of side 516, as shown in FIG. 3B. Thus, when the trigger 518 is squeezed, it will pivot about the longitudinal axis of pivot rod 568 located within the sides 514, 516.

The trigger 518 further preferably includes a hook-shaped safety button engaging member 570 which includes an inner receiving pocket 572 which either hovers above or rests upon the safety button 522 or is received within one of the grooves of the safety button 522, as will be further described with reference to FIGS. 12B-13B.

Turning now to FIGS. 12B-13B, the safety button 522 preferably includes a pair of pins 574, 576. Each of the pins 574, 576 include an engageable end 578, 580, respectively, which protrude through the openings 544 of the sides 514, 516 and are accessible by the operator. Surrounding the pins 574, 576, respectively, are the springs 524, 526. The springs 524, 526 are seated within pockets 545 (FIGS. 6B and 7B) of the openings 544. The pockets 545 have a slightly larger diameter than the exteriormost openings 544 such that the springs 524, 526 received therein abut against a wall 543 within the pockets 545. The safety button 522 further preferably includes three ribs, shown collectively as ribs 582. The ribs 582 include a first side rib 584, a second side rib 586, and a middle rib 588. A first side gap 590 is created between the first side rib 584 and the middle rib 588 and a second side gap 592 is created between the second side rib 586 and the middle rib 588.

When assembled within the handle assembly 512, the safety button 522 is preferably centrally located, and spring biased to be centrally located, within the handle assembly 512 such that the safety button engaging member 570 of the trigger 518 abuts with the middle rib 588 when an attempt to squeeze the trigger 518 is made. However, when the engageable end 578 of the safety button 522 is depressed by an operator, the safety button 522 moves within the handle assembly 512 such that the safety button engaging member 570 of the trigger 518 will fall into the gap 590. Thus, the trigger 518 is now free to be squeezed by the operator. Likewise, when the engageable end 580 of the safety button 522 is depressed by an operator, the safety button 522 moves within the handle assembly 512 such that the safety button engaging member 570 of the trigger 518 will fall into the gap 592 freeing the trigger 518 to be moved by the operator. While the accessibility of the safety button 522 from either side 514 or 516 of the handle assembly 512 provides ease of use to the operator, it is within the scope of this invention to have the safety button accessible from only one side 514 or 516 of the handle assembly 512, which would thus require only one gap and only one pair of ribs in the safety button 522. Pressing the safety button 522 will preferably allow the safety button engaging member 570 to be retained between two adjacent ribs, i.e. ribs 584 and 588 or ribs 588 and 586, but with enough space within either gap 590 or 592 to allow movement of the safety button engaging member 570 during a squeeze of the trigger 518. The purpose of the safety button 522 is to prevent unintentional accidental firing of the device 510. Thus, preferably the safety button 522 is self-centering due to springs 524, 526. After depressing the safety button 522 and releasing the trigger 518 for movement, and after the trigger 518 is squeezed and released by the operator, the safety button 522 will preferably return to its center position, re-locking the trigger 518 from movement. Preferably, the device 510 is a one-time use instrument such that a ferrule 542 cannot be reloaded within the device.

Turning now to FIGS. 14B and 15B, and as additionally shown in FIG. 1B, the adjustment screw 528 is shown which facilitates ferrule loading by the manufacturer. The adjustment screw 528 preferably includes a distal end 594 and a proximal end 596. The distal end 594 may include a bulbous or larger diametered head 598. The proximal end 596 may include a slotted portion 600. A shaft 602 preferably connects the distal end 594 to the proximal end 596. Extending within the adjust screw 528 is a longitudinal bore 604 which extends along the longitudinal axis 606. The bore 604 may have a smaller inner diameter 603 within the head 598 than within the shaft 602. As demonstrated in FIG. 1B, the adjustment screw 528 is connected to a proximal end 532 of the central rod 530, which extends from a distal end 513 of the handle assembly 512. The adjustment screw 528 is preferably completely contained within the handle assembly 512 and is not accessible by the operator. During assembly, the adjustment screw 528 accepts the section 531, preferably threaded, of the proximal end 532 of the central rod 530 so that the length of the central rod 530 may be properly adjusted with respect to the tubular portion 538. The smaller inner diameter 603 is also preferably threaded such that the inner diameter 603 may be threaded to mate with threads on section 531 of rod 530. Turning the adjustment screw 528 after loading the ferrule 542 shortens the rod 530 and allows the distal end 608 of the hammer element 534 to retain the ferrule 542 in the ferrule accepting opening 642 in the tip 540. That is, the proper length of the central rod 530 with respect to the tubular portion 538 helps ensure that the ferrule 542 is retained within the distal end of the device 510. Also, the ability to correct the length of the central rod 530 using the adjustment screw 528 eliminates the need to require very tight tolerances during manufacture of the central rod 530, thus easing the manufacturing process of the device 510.

Turning now to FIGS. 16B-17B, the hammer element 534 is shown. The hammer element 534 includes a distal end 608 and a proximal end 610. The distal end 608 includes the hammer head 612. The hammer head 612 preferably includes a first camming surface 614 which engages with a camming surface on the tip 540 as will be further described. The first camming surface 614 is located on a first side 626 of the hammer element 34. The first camming surface 614 and the first side 626 form an obtuse angle as shown. Located on the second side 628 of the hammer element 534 is a ferrule engaging edge 616. An indent 618 may separate the ferrule engaging edge 616 and an edge 620. Alternatively edge 620 may be removed and replaced with a smooth continuous edge, continuous with second side 628. The hammer element 534 includes a central portion 622 of a selected width which is smaller in width than a proximal portion 624. The smaller width of the central portion 622 allows movement of the hammer head 612 within the tip 540. Each of the first side 626 and second side 628 may comprise a series of planar surfaces as shown in FIG. 17B, with planar sides 630 connecting the first and second sides 626, 628. Thus, the hammer element 534 may comprise a rectangular cross-section. As shown in FIG. 1B, the proximal end 610 of the hammer element 534 is mounted to the distal end 536 of the central rod 530.

Turning now to FIGS. 18B-21B, tip 540 is shown in detail. Tip 540 has a distal end 632 and a proximal end 634. The distal end 632 includes a hammer head receiving portion 636 which includes a second camming surface 638 (shown in FIG. 21B), which abuts with the first camming surface 614 of the hammer head 534 during retraction of the hammer element within the device 510. The second camming surface 638 forms part of a wall of the opening 640 of the tip 540. The opening 640 preferably extends the length of the tip 540 and has a rectangular cross section (as shown in FIG. 18B) throughout most of the tip 540 for receiving the rectangularly shaped hammer element 534. The distal end 632 of the tip 540 further includes a ferrule accepting opening 642 which shares open space with the opening 640. Thus, the opening at the distal end 632, as shown in FIGS. 19B and 20B is generally key-hole shaped. Proximal the ferrule accepting opening 642 is a cutting edge 644 formed on an inner wall of the tip 540 for cutting the suture material as will be further described below.

Alternately, dual hammer heads may be configured to cam in opposite directions against the ferrule during retraction, and said cutting tip may be positioned on one or both of said hammer heads or by virtue of contact between the hammer heads, such that retraction of the hammer heads causes the hammer heads to come together to sever suture after or during crimping. Thus, retraction of at least one hammer head effects both crimping of a ferrule and cutting of suture provided therethrough.

Adjacent the cutting edge 644 is an aperture 646 within the tip 540. The aperture 646 allows the suture material to be threaded through the ferrule 542 from the distal end 632 and exit the aperture 646. The tip 540 preferably includes a proximal portion 648 having a reduced width. A wall 650 is formed between the proximal portion 648 and the hammer head receiving portion 636.

Turning now to FIG. 22B, the distal end 536 of central rod 530 is shown welded to the proximal end 610 of the hammer element 534 at area 652. Notch 654 is shown within the rod 530. Then, the tip 540 is installed upon the hammer element 534. Turning to FIG. 23B, the tubular portion 538 is shown welded or otherwise secured to the tip 540 at or about area 656. The tubular portion 538 overlaps the proximal portion 648 of the tip 540.and abuts the wall 650, so that preferably a smooth continual surface is provided between the tubular portion 538 and the tip 540. Notch 658 within tubular portion 538 coincides with notch 654 in the central rod 530. An anti-rotation feature is provided using the aligned notches 654, 658 during assembly by placing a pin, such as a square pin, into the notch area, thus preventing the rod 530 from rotating within the tubular portion 538. Longitudinal axis 606, which also runs through adjustment screw 528, extends generally through the central rod 530 and tubular portion 538.

FIGS. 24B and 25B show an exemplary ferrule 542 for use within the device 510, and more particularly for placement within the ferrule accepting opening 642 of the tip 540. The ferrule 542 includes a bore 668 which extends the length of the ferrule 542. Ferrule 542 preferably comprises an ovalized outer surface 664. The inner surface 666 of the ferrule 542 will contact the suture material upon compression, as will be described below. The ferrule 542 preferably comprises chamfered ends 660 and 662. The ends may be angled as shown by end 660 or more preferably rounded as shown by end 660. The ferrule 542 is preferably manufactured without burrs of any sort. The material selected for ferrule 542 is preferably annealed titanium, but may be formed from another deformable biocompatible material, such as another non-bioabsorbable material. Alternatively, the ferrule 542 may be formed from a bioabsorbable polymer.

FIGS. 26B-29B describe how the hammer element 534 and tip 540 may cooperate to compress the ferrule 542, secure the suture threads therein, and cut the suture thread ends. The ferrule 542 is shown positioned within the ferrule accepting opening 642 and the suture threads 670 have been threaded through the ferrule 542 and exit the aperture 546 of the tip 540. FIG. 26B shows the hammer head 612 positioned within the tip 540 such that the first camming surface 614 abuts the second camming surface 638. In this initial position, the ferrule engaging edge 616 may abut the ferrule 542 and provide a slight compression of the ferrule 542 for retaining the ferrule 542 within the ferrule accepting opening 642. Turning now to FIG. 27B, as the hammer element 534 is drawn in the direction indicated by arrow 672, the hammer head 612 draws the first camming surface 614 along the second camming surface 638. In doing so, the hammer head 612 is brought closer to the ferrule 542 such that the ferrule engaging edge 616 begins to crimp or compress the ferrule 642. Turning to FIG. 28B, with the hammer element 534 continually drawn in the direction indicated by arrow 672, the first camming surface 614 is no longer in contact with the second camming surface 638, but the first side 626 abuts the inner surface of the opening 640 within tip 540 such that the ferrule engaging edge 616 continues to crimp the ferrule 542. As shown in FIG. 29B, after the hammer element 534 has been moved in the direction indicated by arrow 672 to completely crimp the ferrule 542, the ferrule engaging edge 616 moves towards the cutting edge 644 of the tip 540 until the suture threads 670 are trapped between the hammer head 612 and the cutting edge 644. A small amount of pressure from the hammer head 612 upon the cutting edge 644 will release the ends of the suture threads 670 as shown. Thus, the ferrule 542 is crimped and the suture ends are cut in one step. Additionally, in this embodiment the hammer element 534 does not contain the cutting edge, and therefore there is no risk of providing sharp edges to the ferrule 542 which will remain in the suture location.

FIGS. 30B-34B show one embodiment of a suture loading assembly 674 for assisting an operator in threading the suture threads 670 through the ferrule 542 after a suturing operation and before a suture securing operation. The suture loading assembly 674 preferably includes a body 676 from which extends a flexible loop 678, preferably made from suture material 686 or wire, such as stainless steel wire, and a cap 680. The body 676, as shown in FIG. 34B, includes a distal end 682 from which the loop 678 exits, and a proximal end 684. The body 676 further preferably includes a bore 688 (FIG. 31B) containing the suture material 686 from which the loop 678 is formed. As further shown in FIG. 31B, the body 676 further preferably comprises a step 690 for abutting with a stopping surface 692 within the cap 680. Additionally, the body 676 further includes an attaching member 694, which may have a clip-like shape as shown with a pair of legs 696, 698. The legs 696 and 698 preferably define a rounded receiving pocket 700 for receiving the tubular portion 538.

Because of the small size of the tubular portion 538, and thus the body 676, the suture loading assembly 674 further preferably includes the cap 680 for easy grasping and operating by an operator. The cap 680 includes openings 702 and 704 for receiving the body 676 and attaching member 694. The cap 680 further preferably includes sides 706 which have indents 708 for ease in grasping.

The suture loading assembly 674 is preferably pre-assembled upon the device 510 by the manufacturer. During assembly, the suture loading assembly 674 is preferably secured to the tubular portion 538 by inserting the tubular portion 538 into the opening 704 of the cap 680 and snapping the attaching member 694 onto the tubular portion 538. The loop 678 (which may be much longer than what is shown) may then be pushed into the opening 646 in the tip 540 and threaded through the ferrule 542 which is preloaded within the tip 540. Thus, a portion of the loop 678 will remain extended through the ferrule 542 and out the distal end 632 of the tip 540. Alternatively, the loop 678 could be threaded through the ferrule 542 in the manner described and then the suture loading assembly could be secured to the tubular portion 538.

When a suturing operation has been completed, and it is time to utilize the crimping and cutting device 510 for securing the suture, the ends of the suture material 670 may be simply threaded through the large opening provided in the loop 678. Then, the operator may grasp the cap 680, such as at indents 708, and then the operator may pull the suture loading assembly 674 in a proximal direction, towards the handle assembly 512. In doing so, the loop 678, which is flexible and collapsible, will pull the suture material 670 through the ferrule 542 and out the opening 646 in the tip 540. Because the suture material 670 is likely to be wet and slippery following the suture operation, the ability to thread the suture material 670 through the ferrule 542 using the suture loading assembly 674 eliminates any tedious operational steps.

Turning now to FIGS. 35B-38B, another embodiment of a suture loading assembly is shown. The suture loading assembly 750 is similar in use to the suture loading assembly 774, but embodies a slightly different design. The suture loading assembly 750 includes a wire loop 752 made of wire 753, a body 754, and a plug 756. During assembly, ends 758 of wire loop 752 may be trimmed at location 760 after installing plug 756 so that the wire loop 752 ends flush with a proximal end 762 of the body 754. The wire loop 752 preferably includes a tapered distal end 764, a widest portion 766, and a cross-over portion 768 where the wire 753 crosses over itself prior to running parallel into the body 754.

The body 754 includes a tapered nose section 770 having a distal end 772 and an opening 774. The opening 774 receives the wire 753 of the wire loop 752. As shown in FIG. 38B, the opening 774 leads to a longitudinal bore 776 having a main bore 780 with a first inner diameter, distal tapered section 778 having a smaller inner diameter than the first inner diameter, and a proximal bore 782 having a second inner diameter slightly larger than the first inner diameter, such that a stopping surface 784 is provided within the bore 776. During assembly, the plug 756 is inserted into the proximal end 762 of the body 754 for retaining the wire loop 752 within the body 754.

The body 754 further preferably includes an integral attaching member 786 which includes a pair of clip-like legs 788 separated by a slot 790 having a rounded end 792 for receiving the tubular portion 538. Each leg 788 further preferably includes an indented area 794 for ease in grasping.

As with the suture loading assembly 674, the suture loading assembly 750 is preferably pre-assembled upon the device 510 by the manufacturer. During assembly, the suture loading assembly 750 is preferably secured to the tubular portion 538 by inserting the tubular portion 538 into the slot 790 and snapping the attaching member 786 onto the tubular portion 538 with the tubular portion 538 residing in the rounded end 792 of the slot 790. The wire loop 752, which is sufficiently flexible, may be pushed into the opening 646 in the tip 540 and threaded through the ferrule 542 which is preloaded within the tip 540. Thus, a portion of the wire loop 752 will remain extended through the ferrule 542 and out the distal end 632 of the tip 540. Preferably, the wire loop 752 is preformed such that upon its exit through the ferrule 542, it will begin to open up automatically thus creating a stable opening, as opposed to suture material in which the opening may have to be created by separating the thread used in the loop 678 in the suture loading assembly 674. Alternatively, the wire loop 752 could be threaded through the ferrule 542 in the manner described and then the suture loading assembly 750 could be secured to the tubular portion 538. The ends of suture material 670 may be threaded through the large opening provided in the wire loop 752, and the operator may grasp the indented areas 794 and pull the suture loading assembly 752 in a proximal direction, towards the handle assembly, for pulling the suture material 670 through the ferrule 542 as previously described with the operation of the suture loading assembly 674.

FIGS. 39B and 40B show an alternate embodiment of a suture loading assembly 710, where halves of the suture loading assembly 710 are depicted in FIGS. 39B-40B. Looking at the suture loading assembly from a distal location, i.e. from the funnel 712, FIG. 40B shows a left half 714 and FIG. 39B depicts a right half 716. The two halves 714, 716 preferably snap onto the distal end of the device 510 for threading of the ferrule 542. The suture material 670 may be threaded through a funnel 712 created by a joining of the two halves 714, 716. Then, the threads 670 would go directly into the ferrule 542 after being pushed into the funnel 712. After the ferrule 542 is threaded, the wings 718, 720 may be squeezed together to release the suture loading assembly 710 from the distal end of the device 510. Thus, with the suture material 670 threaded through the ferrule 542, the device 510 may be inserted near the suture location (the area where the body was closed by the suture material 670) to secure the ferrule 542 upon the suture material 670.

### EXAMPLES OF SYSTEMS APPLYING MULTIPLE SUTURES ACROSS A WOUND

Other exemplary embodiments described herein may be used to apply multiple sutures across a single wound. Such embodiments may be useful, e.g. for large-bore arteriotomy procedures, among others. The benefits of the above-described design of a single suture delivery devices may thus be improved for large-bore closures by the introduction of the additional suture(s) across the wound and at distinct edge portions.

In one exemplary embodiment the system comprises multiple suturing devices and at least one crimping and cutting device. In such embodiment, a first suturing device may be placed within the wound to apply a first suture on edge portions across the wound site. Subsequently, a second suturing device may be placed in a different position within the wound to apply a second suture across different edge portions of the wound. Then, the crimping and cutting portion may be used to gather suture ends, and the crimping and cutting portion may be located to the wound site to apply a fastener (e.g., a ferrule).

In another exemplary embodiment, a single suturing device may be configured to apply first and second sutures on different edge portions across the wound site. In an exemplary embodiment, e.g. a dual suture delivery device may be configured similarly to single suture delivery devices with the addition of a second needle and ferrule alongside a first needle and ferrule in one or more tissue receiving gaps.

Also, as has been described above, suture may be looped from one ferrule, through the device, e.g. through a capture window on the device body, and back to a second ferrule. In exemplary embodiments related to a dual suture delivery device, a first suture end at a first ferrule within a first tissue receiving gap extends not to a second ferrule in a second tissue receiving gap that is longitudinally aligned with the first ferrule, but instead to a third ferrule in the second tissue receiving gap that is adjacent the second ferrule, the third ferrule not longitudinally aligned with the first ferrule. In this way, when placed across the wound, first and second sutures will cross each other and tend to draw the edges of the wound toward the cross point of the first and second sutures within the wound.

FIG. 41 illustrates an example of a large-bore arterial wound, shown generally at 400, wherein two sutures 402 and 404 cross each other within the wound after being placed by one or more suture delivery devices as described herein.

In a first methodology, a first suturing device may place suture 402 within the wound followed by insertion and rotation of a second suturing device to place suture 404 within the wound at an angle relative to the first suture 402. FIG. 42 illustrates a top elevation view of a large-bore arterial wound 400, with a second suturing device 406 placing a second suture 404 (not shown in FIG. 42) across different edge portions relative to the edge portions used to place a first suture 402.

Note that a sheath portion 408 has been retracted, e.g. by use of a lever on the device body (see 410 in FIGS. 43 and 44), wingnut on the device body or shaft (see 412 in FIG. 45) or other pull portion for the sheath portion 408. An operative portion 414 of sheath 408 is shown in a first portion that exposes a first tissue receiving gap (on the underside of the operative portion 414 such that the physician may pull back to engage tissue in the first tissue receiving gap to withdraw a ferrule for the second suture 404 through the wound edge portion. Actuation of the lever 410 or wingnut 412 operates to rotate that operative portion to cover the first tissue receiving gap (which is on the backside of the suturing device in FIG. 42) to expose the second tissue receiving gap (which is just under the operative portion of the sheath but on the top side of the suturing device in FIG. 42). Referring to FIG. 42, the surgeon would then push the device forward to secure the last suture end for suture 404 (see FIG. 41), resulting in the illustration at FIG. 41. FIG. 46 illustrates the end result of gathering the plural (in this case four) suture ends through a crimping and cutting device to secure a crimped ferrule 416 over the approximated wound 400.

Referring again to FIG. 44 a second methodology, a single, multi-suture delivery device 420 may be used to place plural sutures sequentially or simultaneously. FIG. 42 illustrates a perspective view of an exemplary multi-suture delivery device 420. Other than the sheath lever 410 for the operative portion 414 of the sheath 408 (described immediately above), each tissue receiving gap 422, 424 includes plural needles in a proximal end portion thereof and corresponding plural ferrules in a distal end portion thereof (in exemplary embodiments, in exactly the same manner as described above with regard to single suture delivery devices with the exception that multiple components are used alongside one another). Also, a suture delivery window 426 shows two leads for a first suture 402 and two leads for a second suture 404, which window will show what suture ends have been successfully captured through tissue upon withdrawal of the device (an unsuccessful capture will not result in a suture end being pulled through the window).

FIG. 47 illustrates an exemplary first deployment of needles (see one needle 428 in FIG. 47) at a lower, first tissue receiving gap 422. FIG. 48 then illustrates deployment of third and fourth needles 432, 434 across a second, upper tissue receiving gap 424. FIG. 49 illustrates removal of the device with sutures 402 and 404 approximating the wound 436. FIG. 50 illustrates successful capture of both strands of suture via a vacant suture window 426. Also, FIG. 51 shows an alternate embodiment facilitating deployment of dual needles across each tissue gap simultaneously.

FIG. 52 illustrates an exemplary embodiment, wherein plural sutures 402, 404 are loaded through a cinching device 438 that is configured to receive the plural sutures. FIG. 53 illustrates crimping of the plural sutures inside a ferrule, wherein the ferrule experiences crimping forces from different sides (e.g., the dual hammer embodiment discussed above). FIG. 54 illustrates an alternate embodiment using plural ferrules 416 with plural crimping.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A crimping and cutting device comprising:
at least one hammer head having a first side and an opposite second side, and a ferrule engaging edge located on the second side;
a tip having a distal end and a proximal end, the tip having a hammer head opening for receiving the hammer head, the hammer head opening extending from the distal end of the tip to the proximal end of the tip, the tip further having a ferrule accepting opening near the distal end of the tip, and at least one camming surface configured to apply pressure to the ferrule through the at least one hammer to crimp suture within said ferrule upon retraction of the at least one hammer head, wherein the at least one hammer is further configured to sever suture proximal of said ferrule during or after crimping of said ferrule

2. The crimping and cutting device of claim 1 wherein the hammer head further comprises a first camming surface located on the first side of the hammer head and the tip comprises a second camming surface near the distal end of the tip and opposite the ferrule accepting opening.

3. The crimping and cutting device of claim 2 wherein movement of the hammer head in a proximal direction directs the hammer head towards the ferrule accepting opening.

4. The crimping and cutting device of claim 2 wherein the second camming surface forms a wall of the hammer head opening and flares outwardly towards the distal end of the tip.

5. The crimping and cutting device of claim 2 wherein the first camming surface and the second camming surface abut flushly when the hammer head is at the distal end of the tip.

6. The crimping and cutting device of claim 2 wherein the first camming surface does not abut the second camming surface when the hammer head is pulled proximally of the ferrule accepting opening.

7. The crimping and cutting device of claim 1 wherein the tip further comprises an aperture between the distal end and the proximal end of the tip, the cutting edge located distally of the aperture.

8. The crimping and cutting device of claim 1 wherein proximal movement of the hammer head within the tip causes the ferrule engaging edge of the hammer head to contact a cutting edge after the hammer head has passed the ferrule accepting opening of the tip.

9. The crimping and cutting device of claim 1 wherein the tip comprises a distal end outer diameter and a proximal end outer diameter, wherein the outer diameter of the distal end is greater than the outer diameter of the proximal end.

10. The crimping and cutting device of claim 1 further comprising a handle assembly having a trigger, wherein activation of the trigger draws the hammer head proximally within the tip.

11. The crimping and cutting device of claim 10 further comprising a safety button, wherein the trigger cannot be activated until the safety button is depressed.

12. The crimping and cutting device of claim 10 further comprising an adjustment screw within the handle assembly, wherein a length of a central rod connecting the hammer head to the handle assembly is adjustable by the adjustment screw.

13. The crimping and cutting device of claim 1 further comprising a tubular portion extending from the proximal end of the tip, the device further comprising a suture loading assembly mounted on the tubular portion and slidable along the tubular portion.

14. The crimping and cutting device of claim 1 wherein the suture loading assembly comprises a loop threadable through the ferrule accepting opening.

15. A method of securing suture material using a crimping and cutting device, the method comprising:
threading the suture material through a ferrule in the device;
moving a hammer head proximally through a tip of the device to crimp the ferrule;
continuing to move the hammer head proximally after the ferrule has been crimped entrapping the suture material within the ferrule;
abutting the hammer head against a cutting edge within the tip, capturing the suture material therebetween; and,
applying pressure with the hammer head against the cutting edge until the suture material is cut.

16. The method of claim 15 wherein moving the hammer head proximally comprises squeezing a trigger on a handle assembly of the crimping and cutting device.

17. The method of claim 16 wherein a safety button is depressed on the handle assembly prior to squeezing the trigger.

18. The method of claim 15 wherein threading the suture material through a ferrule comprises threading the suture material through a flexible loop extending from a distal end of the ferrule and pulling the flexible loop proximally until the suture material is threaded through the ferrule.

19. The method of claim 15 wherein pulling the flexible loop proximally comprises sliding a suture loading assembly, which is mounted on a tubular portion of the cutting and crimping device and to which the flexible loop is attached, proximally along the tubular portion.

20. A suture applying device for applying plural sutures across a percutaneous wound, comprising:
a first tissue receiving gap, the first tissue receiving gap and a second tissue receiving gap opposite and longitudinally displaced from the first tissue receiving gap;
at least two needles configured to extend distally across said first tissue receiving gap to capture ends of separate sutures attached to ferrules on a distal side of said first tissue gap, wherein retraction of said at least two needles removes said suture ends and ferrules from said distal side of said first tissue receiving gap.
at least two additional needles configured to extend distally across said second tissue receiving gap to capture opposite ends of said separate sutures attached to ferrules on a distal side of said second tissue receiving gap, wherein retraction of said at least two needles removes said suture ends and ferrules from said distal side of said second tissue receiving gap.
